(19) United States
(12) Patent Application Publication
(10) Pub. No.: US 2008/0193536 A1
(43) Pub. Date: Aug. 14, 2008

(54) Cell-Laden Hydrogels

(76) Inventors: Alireza Khademhosseini, Cambridge, MA (US); Jeffrey M. Karp, Brookline, MA (US); Omid C. Farokhzad, Chestnut Hill, MA (US); Robert S. Langer, Newton, MA (US)

Correspondence Address: CHOATE, HALL & STEWART LLP, TWO INTERNATIONAL PLACE, BOSTON, MA, 02110, US

(21) Appl. No.: 11/838,752

(22) Filed: Aug. 14, 2007

Related U.S. Application Data
(60) Provisional application No. 60/837,870, filed on Aug. 14, 2006.
(60) Provisional application No. 60/837,875, filed on Aug. 14, 2006.

(51) Int. Cl. A61K 9/00; A61K 35/12

(52) U.S. Cl. 424/486; 424/484; 424/93.7; 424/488

(57) ABSTRACT

The present invention provides cell-laden hydrogels and hydrogel assemblies thereof for use in tissue engineering. The invention provides microscale hydrogels (i.e. microgels) having greatest dimensions ranging between about 1 μm and 1000 μm. The present invention provides methods of producing inventive hydrogels and hydrogel assemblies and pharmaceutical compositions thereof.

### RELATED APPLICATIONS

The present application is related to and claims priority under 35 U.S.C. §119(e) to U.S. provisional patent applications, U.S. Ser. No. 60/837,870, filed Aug. 14, 2006 (“the '870 application”), and U.S. Ser. No. 60/837,875, filed Aug. 14, 2006 (“the '875 application”). The entire contents of the '870 application and the '875 application are incorporated herein by reference.

### GOVERNMENT SUPPORT

The United States Government has provided grant support utilized in the development of the present invention. In particular, the National Institutes of Health (contract number HL 60435) have supported development of this invention. The United States Government has certain rights in the invention.

### BACKGROUND OF THE INVENTION

Traditionally, approaches to restore tissue function have involved organ donation. However, despite attempts to encourage organ donations, there is a shortage of transplantable human tissues. Currently more than 74,000 patients in the United States are awaiting organ transplantation, while only 21,000 people receive transplants annually. Tissue engineering may provide a possible solution to alleviate the current shortage of organ donors. Tissue engineering is an interdisciplinary field that applies the principles of engineering and life sciences to develop biological substitutes, typically composed of biological and synthetic components that restore, maintain or improve tissue function. Tissue engineered products would provide a life-long therapy and would greatly reduce the hospitalization and health care costs associated with drug therapy, while simultaneously enhancing the patients' quality of life. Tissue engineering approaches have been used to create a number of biological substitutes such as bone, cardiac, smooth muscle, pancreatic, liver, tooth, retina, and skin tissues. Although tissue engineering has been relatively successful for tissues such as skin and cartilage, complex three-dimensional (3D) tissues having precisely-defined matrix properties and spatial organization of cells have not yet been generated.

Therefore, there is a strong need in the art for shape- and size-controlled hydrogels in which cells have been encapsulated with homogeneous cell distribution at various viable cell densities, for methods of producing such hydrogels in the form of harvestable free units, and for tissue engineering applications in which such hydrogels mimic the architectural intricacies of physiological cell-cell interactions.

### SUMMARY OF THE INVENTION

The present invention provides three-dimensional (3D) microscale hydrogels (i.e. microgels) of controlled shapes and sizes in the form of harvestable, free-standing units. Cell-laden hydrogels generally comprise cells and at least one polymer capable of forming a hydrogel.

In general, cells are encapsulated within a hydrogel by mixing a cell suspension with a precursor solution and crosslinking or polymerizing the resulting mixture. In accordance with the present invention, a precursor solution comprises one or more polymers and/or polymer precursors (e.g., monomers, oligomers, etc.) and, optionally, one or more cells. Any polymer that, upon crosslinking and/or polymerization, is capable of forming a hydrogel can be used in accordance with the present invention. In some embodiments, the percent of polymer in a precursor solution that is suitable for forming hydrogels in accordance with the present invention ranges between about 1% w/w and about 40% w/w.

In some embodiments, a polymer to be included in a precursor solution in accordance with the present invention may be a natural polymer, such as a carbohydrate, protein, nucleic acid, lipid, etc. In some embodiments, natural polymers may be synthetically manufactured.

In some embodiments, a polymer to be included in a precursor solution in accordance with the present invention may be a carbohydrate. In certain embodiments, a carbohydrate polymer is a glycosaminoglycan, including, but not limited to, hyaluronic acid (HA), chondroitin sulphate, dermatan sulphate, keratan sulphate, and/or heparan sulphate. In some embodiments, carbohydrate polymer is alginate, chitosan, heparin, agarose, dextran, cellulose, and/or derivatives thereof.

In some embodiments, a polymer in to be included in a precursor solution in accordance with the present invention may be a protein or peptide. In certain embodiments, a protein polymer is collagen, elastin, fibrin, albumin, poly(amino acid), glycoprotein, and/or antibody.

In some embodiments, polymers to be included in a precursor solution in accordance with the present invention may be synthetic polymers. In some embodiments, polymers to be included in a precursor solution in accordance with the present invention are hydrophilic. In some embodiments, polymers to be included in a precursor solution in accordance with the present invention are chemically neutral. Neutral synthetic polymers can be generated from derivatives of poly(ethylene glycol) (PEG), poly(hydroxyethyl methacrylate) (PHEMA), and poly(vinyl alcohol) (PVA).

In general, cells to be used in accordance with the present invention are any types of cells. In general, the cells should be viable when encapsulated within hydrogels. In some embodiments, cells that can be encapsulated within hydrogels in accordance with the present invention include, but are not limited to, mammalian cells (e.g. human cells, primate cells, mammalian cells, rodent cells, etc.), avian cells, fish cells, insect cells, plant cells, fungal cells, bacterial cells, and hybrid cells. In some embodiments, exemplary cells that can be encapsulated within hydrogels include stem cells, totipotent cells, pluripotent cells, and/or embryonic stem cells. In some embodiments, exemplary cells that can be encapsulated within hydrogels in accordance with the present invention include, but are not limited to, primary cells and/or cell lines from any tissue. In some embodiments, the percent of cells in a precursor solution that is suitable for forming hydrogels in accordance with the present invention ranges between about 0.1% w/w and about 80% w/w.

In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, the conditions under which cells are encapsulated within hydrogels are altered in order to maximize cell viability.

In some embodiments, a single hydrogel comprises a population of identical cells and/or cell types. In some embodiments, a single hydrogel comprises a population of cells and/or cell types that are not identical. In some embodiments, a single hydrogel may comprise at least two different types of cells. In some embodiments, a single hydrogel may comprise 3, 4, 5, 10, or more types of cells.

The present invention provides methods for producing cell-laden hydrogels in accordance with the present invention. Inventive cell-laden hydrogels may be manufactured using any available method. In some embodiments, cells are suspended in a polymer precursor solution and molded using a stamp. Subsequently, the polymer precursor solution is crosslinked and/or polymerized to form a gel. The mold is then removed to generate an array of micromolded hydrogels that can be harvested into a solution using a simple wash.

In some embodiments, inventive methods utilize a stamp in which the precursor solution is molded. In some embodiments, stamps comprise wells and intervening ridges. In some embodiments, the wells have a greatest dimension ranging between approximately 0.5 μm and approximately 5000 μm. In some embodiments, stamps have a greatest dimension ranging between about 50 μm and several meters. In some embodiments, stamps can be of a size and shape that is compatible with particular laboratory equipment that is used in the production of hydrogels (e.g. a stamp may be of a shape and size that allows the stamp to be mounted on a standard glass slide).

In some embodiments, stamps are manufactured by curing (e.g. heat-curing) a prepolymer on a master stamp. In some embodiments, a master stamp may comprise a silicone surface, metal surface, plastic surface, ceramic surface, glass surface, etc. After curing, the resulting stamp is peeled off of the master stamp.

In some embodiments, stamps can be made of any material. In general, the surface of the stamp which is exposed to the precursor solution should be hydrophilic. If the stamp is made of a hydrophobic substance, it can be made hydrophilic by any method known in the art (e.g. plasma cleaning, chemical derivitization of surface, etc.). In specific embodiments, the stamp can be made of hydrophilic poly(dimethylsiloxane) (PDMS) and/or PMDS that has been surface-treated such that it is hydrophilic.

After the precursor solution has been introduced into the wells, the precursor solution is subjected to conditions that induce crosslinking and/or polymerization. Any crosslinking method known in the art can be utilized, including, but not limited to, photocrosslinking, chemical crosslinking mechanisms, physical crosslinking mechanisms, irradiative crosslinking mechanisms, thermal crosslinking mechanisms, ionic crosslinking mechanisms, and the like.

In some embodiments, inventive cell-laden microscale hydrogels (i.e. microgels) can be assembled into hydrogel assemblies comprising a plurality of individual microgels. Among other things, such hydrogel assemblies can be useful for forming tissues and/or organs for medical therapies. In general, microgels are large enough to encapsulate at least one cell. In some embodiments, microgels have a greatest dimension ranging between approximately 1.0 μm and approximately 1000 μm

In some embodiments, microgels stack together in a linear fashion to form chain-like hydrogel assemblies. In some embodiments, microgels stack together in a two-dimensional fashion to form sheet-like hydrogel assemblies. In some embodiments, microgels stack together in a three-dimensional fashion to form cuboid, rectangular, spherical, conical, pyramid-like, cylindrical, tubular, ring-shaped, tetrahedral, hexagonal, octagonal, other regularly-shaped, and/or irregularly-shaped hydrogel assemblies.

Hydrogel assemblies in accordance with the present invention can be formed using any available method. In some embodiments, hydrogel assemblies are formed by self-assembly. In specific embodiments, surface modification of hydrogels can be used as a method of initiating self-assembly. In some embodiments, microgel shape can be controlled so that the microgels can “self-assemble” into tissues using geometric lock-and-key mechanisms. In some embodiments, microgels may be assembled into hydrogel assemblies using surface tension mechanisms. In some embodiments, a micromanipulator can be utilized to manually assemble microgels into hydrogel assemblies.

Once hydrogels assemblies are formed, it is possible to further induce their crosslinking to coalesce the individual microgels to one another. This can be accomplished using a multistep crosslinking approach in which the gels are partially crosslinked, assembled into hydrogel assemblies, and further crosslinked.

In some embodiments, all of the individual microgels of the plurality of microgels are of identical composition, shape, size, etc. In some embodiments, some of the microgels of the plurality of microgels have one particular composition, shape, size, etc., and others of the microgels of the plurality of microgels have another particular composition, shape, size, etc.

Cell-laden hydrogels and/or hydrogel assemblies in accordance with the present invention may be used for tissue engineering applications. In some embodiments, tissue engineering aims to replace, repair, and/or regenerate tissue and/or organ function or to create artificial tissues and organs for transplantation. In general, scaffolds used in tissue engineering (e.g. hydrogel scaffolds) mimic the natural extracellular matrix (ECM) and provide support for cell adhesion, migration, and proliferation.

In some embodiments, hydrogels and/or hydrogel assemblies in accordance with the present invention to be used for tissue engineering applications can be formed in situ, enabling the polymer to conform to the shape of the implantation site.

In some embodiments, methods described herein may be used to construct complex delivery devices capable of precisely defined release profiles. This could be achieved through combining drugs or drug delivery devices (i.e. nanoparticles or microparticles) with inventive cell-laden hydrogels and/or hydrogel assemblies and using these to construct more complex drug delivery systems.

Cell-laden hydrogels, hydrogel assemblies, and/or precursor solution for in situ hydrogel formation may be administered by any route. In some embodiments, compositions of the present invention are administered by a variety of routes, including direct administration to an affected site. For example, inventive compositions may be administered locally near a site which is in need of tissue regeneration. Local administration may be achieved via injection of hydrogel precursor solution directly to a site in need of tissue regrowth followed by crosslinking, such that a cell-laden hydrogel is formed in situ.

The invention provides a variety of kits comprising one or more of the hydrogels and/or hydrogel assemblies of the invention. According to certain embodiments of the invention, a kit may include, for example, (i) a precursor solution comprising a cell, a polymer, and a crosslinking initiator; and (ii) instructions for forming a hydrogel from the precursor solution. In some embodiments, a kit may include, for example, (i) a precursor solution comprising a cell, a polymer, and a crosslinking initiator; and (ii) instructions for administering the precursor solution to a patient in need thereof and performing a crosslinking step such that a cell-laden hydrogel is formed in situ. In some embodiments, a kit may include, for example, (i) a plurality of microgels, each comprising a cell and at least one polymer; and (ii) instructions for forming a hydrogel assembly from the plurality of microgels.

This application refers to various issued patents, published patent applications, journal articles, and other publications, all of which are incorporated herein by reference.

### BRIEF DESCRIPTION OF THE DRAWING

FIG. 1: Charge and hydrophobic/hydrophilic interactions facilitate self-assembly. Hydrophilic microgels of opposite charges (shown here are chitosan and hyaluronic acid) are incubated in a hydrophobic bath to induce self-assembly.

FIG. 2: High throughput surface modification of hydrogels. (Left) Microfluidic channels are first placed over the formed micromolded materials. (Right) Microfluidic channels can be arranged in a number of permutations and orthogonally to achieve multiple simultaneous surface modifications.

FIG. 3: Lock-and-key assembly of microscale hydrogels comprising various cell types for tissue fabrication.

FIG. 4: Seven microgels have assembled in a hydrophobic solution to form a hydrogel assembly.

FIG. 5: Hydrogel assemblies at various time points during formation. 200 μm squares were allowed to assemble while stirring. Various timepoints are shown: (A) 30 seconds, (B) 60 seconds, (C) 120 seconds, (D) 150 seconds, (E) 180 seconds.

FIG. 6: Microgels containing individual or multiple cell types can be used for scale-up of bioreactors or for generating 3D tissues with spatial control of various cells or tissue architecture.

FIG. 7: General scheme of bottom-up assembly process. Step 1: Individual functional cardiac myofibrils are generated. Step 2: Individual cardiac myofibrils are encapsulated within microgels. Step 3: Individual encapsulated cardiac myofibrils assembled into a desired 3D structure and induced to interconnect and join into a single functional tissue.

FIG. 8: Exemplary process of cell encapsulation and microgel formation. (A) Cells are suspended in prepolymer solution and deposited onto a plasma-cleaned PDMS pattern. (B) A PDMS coverslide is placed on top, forming a reversible watertight seal. (C) Polymer liquid is photopolymerized via exposure to UV light. (D) The PDMS coverslide is lifted, (E) removing the microgels which are then (F) hydrated and harvested.

FIG. 9: Versatility in microgel shapes. Microgels can be molded into various shapes: square prisms (A), (B), disks (C), and strings (D), stained with trypan blue to facilitate visualization, using different prepolymer solutions: (A) MeHA and (B), (C), (D) PEGDA. (E) Comb-like structure of PEG hydrogel. (F) PEG hydrogel discs with encapsulated live cells.

FIG. 10: Optimization for initial cell viability. Varied parameters include macromer (HA) concentrations, photoinitiator (Irg for Irgacure) concentrations, and UV exposure durations. Cell viability was observed to be optimal (92%±4%) for 5 wt % HA in PBS with 1 wt % photoinitiator. Error bars indicate standard deviation values for n=3.

FIG. 11: Cell encapsulation, viability, and distribution. Cells were encapsulated in MeHA (A) and PEGDA (C) microgels and stained with viability markers ((B), (D): ethidium homodimer permeabilizes dead cells, showing up as red; calcein AM is metabolized by live cells, showing up as green). (E) Confocal imaging shows an even distribution of cells (rhodamine-stained) throughout the depth of microgels.

FIG. 12: Harvesting microgels. Removal of the PDMS coverslide following UV exposure yields a uniform array of HA microgels with cells encapsulated inside (A). The subsequent hydration allows these microgels to be dislodged and suspended in solution (C). Viability stains (B), (D) show >85% viability.

FIG. 13: Variation in cell density. (A)-(D) Cell density in microgels can be finely controlled. Variations shown range from (A), (B) 5×10⁷ cells/ml to (C), (D) 20×10⁷ cells/ml HA prepolymer solution. Viability stains (B), (D) show >85% viability.

FIG. 14: Microgel arrangement and assembly. Microgels were created with 3T3 cells stained either with a red or green stain and then assembled into more complex macrostructures. Left: Rhodamine (red) and FITC (green) stained 3T3 cells encapsulated in separate hyaluronic acid methacrylate hydrogel microsquares arranged in an alternating checkerboard pattern. Right: Green stained 3T3 cells encapsulated in a hydrogel square that has subsequently been polymerized in a drop of red stained 3T3 cells. Scale bar represents 200 μm.

### DEFINITIONS

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, genetically-engineered animal, and/or a clone.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Biocompatible: As used herein, the term “biocompatible” refers to substances that are not toxic to cells. In some embodiments, a substance is considered to be “biocompatible” if its addition to cells in vitro results in less than or equal to approximately 20% cell death. In some embodiments, a substance is considered to be “biocompatible” if its addition to cells in vivo does not induce inflammation and/or other adverse effects in vivo.

Biodegradable: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Hydrogel: As used herein, the term “hydrogel” refers to a three-dimensional (3D) crosslinked network of hydrophilic polymers that swell in water. In some embodiments, water can penetrate in between the polymer chains of the polymer network, subsequently causing swelling and the formation of a hydrogel. In general, hydrogels are superabsorbent. For example, in some embodiments, hydrogels can contain over 50%, over 60%, over 70%, over 80%, over 90%, over 91%, over 92%, over 93%, over 94%, over 95%, over 96%, over 97%, over 98%, over 99%, or more water. In some embodiments, cells and/or therapeutic agents to be delivered can be encapsulated within hydrogels. Typically, cells are encapsulated within hydrogels through mixing a cell suspension with a precursor solution (i.e. a solution comprising a polymer suitable for hydrogel formation) and crosslinking the resulting network using any available means for crosslinking. In some embodiments, a plurality of hydrogels can be assembled together to form a hydrogel assembly.

In vitro: As used herein, the term “in vitro” refers to events that occur in an artificial environment, e.g., in a test tube or reaction vessel, in cell culture, etc., rather than within an organism (e.g. animal, plant, and/or microbe).

In vivo: As used herein, the term “in vivo” refers to events that occur within an organism (e.g. animal, plant, and/or microbe).

Microgel: As used herein, the term “microgel” refers to a microscale hydrogel. In general, microgels are large enough to encapsulate at least one cell. Microgels typically have a greatest dimension of less than approximately 5000 μm. In some embodiments, microgels have a greatest dimension of less than approximately 5000 μm, less than approximately 1000 μm, less than approximately 100 μm, less than approximately 10 μm, or smaller. In some embodiments, microgels have a greatest dimension of approximately 1.0 μm, approximately 5 μm, approximately 10 μm, approximately 50 μm, approximately 100 μm, approximately 250 μm, approximately 500 μm, approximately 750 μm, or approximately 1000 μm. In some embodiments, a plurality of microgels can be assembled together to form a hydrogel assembly.

Precursor solution: As used herein, the term “precursor solution” refers to a solution comprising one or more polymers and/or polymer precursors (e.g. monomers, oligomers, etc.) that can be induced to form a hydrogel and, optionally, one or more cells. In general, a precursor solution is induced to form a hydrogel via crosslinking and/or polymerization of the polymers within the precursor solution.

Subject: As used herein, the term “subject” or “patient” refers to any organism to which a composition of this invention may be administered, e.g., for experimental, diagnostic, and/or therapeutic purposes. Typical subjects include animals (e.g., mammals such as mice, rats, rabbits, non-human primates, and humans) and/or plants.

Therapeutically effective amount: As used herein, the term “therapeutically effective amount” means an amount of a therapeutic and/or diagnostic agent (e.g., inventive targeted particle) that is sufficient, when administered to a subject suffering from or susceptible to a disease, disorder, and/or condition, to treat and/or diagnose the disease, disorder, and/or condition.

Therapeutic agent: As used herein, the phrase “therapeutic agent” refers to any agent that, when administered to a subject, has a therapeutic, diagnostic, and/or prophylactic effect and/or elicits a desired biological and/or pharmacological effect.

### DETAILED DESCRIPTION OF CERTAIN EMBODIMENTS OF THE INVENTION

The present invention provides three-dimensional (3D) microscale hydrogels (i.e. microgels) of controlled shapes and sizes in the form of harvestable, free-standing units. The hydrogels generally comprise cells and at least one polymer capable of forming a hydrogel. The present invention provides hydrogel assemblies, in which microgels are assembled into higher-order 3D structures comprising a plurality of individual microgels. The present invention provides therapeutic applications for hydrogels and hydrogel assemblies, such as tissue regeneration. The present invention provides methods of producing inventive microscale hydrogels (i.e. microgels) and hydrogel assemblies.

### Hydrogels

In general, hydrogels comprise three-dimensional (3D) crosslinked networks of hydrophilic polymers that swell in water. Water can penetrate in between the polymer chains of the polymer network, subsequently causing swelling and the formation of a hydrogel (Langer and Peppas, 2003, AICHE J., 49:2990). Hydrogels are superabsorbent (e.g. they can contain over 99% water) and possess a degree of flexibility very similar to natural tissue, due to their significant water content.

Encapsulation of cells within hydrogels has been proposed as a method of enabling the scalable expansion of anchorage dependant cells within stirred bioreactors. However, the immobilization of cells within larger structures decreases the viability of cells in the center of these structures due to lack of appropriate levels of oxygen and nutrient (Pathak and Hubbell, 1992, supra; and Godbey, 2002, Ann. N.Y. Acad. Sci., 961:10). Spherical microcapsules with high surface area to volume ratios and coated annuli of cells immobilized within polymers have therefore been generated to overcome transport difficulties (Pathak and Hubbell, 1992, supra). Most approaches to generate such structures have been based on spherical structures because of the available technologies to generate microscale spheres based on emulsification (Dang et al., 2002, supra) or shear-induced droplet formation from syringes (Lahooti et al., 2000, supra). These approaches have been shown to be capable of forming spherical cell-laden (microscale hydrogels) microgels of controlled sizes; however, they are not amenable to generation of other shapes, defined control over shape, or production of homogeneous cell-laden hydrogels.

Recently, photolithography (Koh et al., 2003, Anal. Chem., 75:5783; Liu and Bhatia, 2002, Biomed. Microdev., 4:257; and Koh et al., 2002, Langmuir, 18:2459) and soft lithography (Tang et al., 2003, J. Am. Chem. Soc., 125:12988) have been used to encapsulate live cells within small units of polymeric hydrogels anchored onto two-dimensional (2D) surfaces. Although these systems offer great potential for diagnostic and cell screening applications, diffusion of nutrients and metabolites can occur only at one interface, which may compromise long term cell culture.

Typically, cells are encapsulated within hydrogels through mixing a cell suspension with a precursor solution (i.e., a solution comprising a polymer suitable for hydrogel formation) and crosslinking the resulting network. The crosslinking reaction may be controlled by a variety of environmental factors such as temperature, pH, and/or the addition of chelating ions. In some embodiments, hydrogels can be photopolymerized in the presence of photoinitiators via exposure to ultraviolet (UV) light (Scranton and Bea, Photopolymerization fundamentals and applications, ACS Publishers, 1996). Hydrogels comprising natural polymers (e.g., fibrin [Sakiyama et al, 1999, FASEB J., 13:2214], hyaluronic acid (HA) [Burdick et al., 2005, Biomacromolecules, 6:386], agarose [Khademhosseini et al., 2005, supra]) and synthetic polymers (e.g., poly(ethylene glycol) (PEG) [Halstenberg et al., 2002, Biomacromolecules, 3:710; and Elisseeff et al., J. Biomed. Mater. Res., 51:164]) have been used to encapsulate cells. For example, photopolymerized PEG diacrylate hydrogels, have been explored for the transplantation of islets of Langerhans for development of a bioartificial endocrine pancreas (Pathak et al., 1992, J. Am. Chem. Soc., 114:8311; Cruise et al., 1999, Cell Transplant, 8:293; and Sawhney et al., 1993, Biomaterials, 14:1008). Similarly, photopolymerized hyaluronic hydrogels have been investigated as potential implantable/injectable cell delivery vehicles for cartilage regeneration (Ki Hyun Bae and Tae Govan, 2006, Biotechnol. Prog., 22:297).

The present invention encompasses the recognition that cell encapsulation within free micro-sized units of hydrogel (i.e., in suspension) may be advantageous not only for immunoisolation and bioreactor applications where long term cell culture is imperative, but also for tissue engineering. Such systems allow creation of micro-sized units of hydrogel that can be assembled into tissue engineering constructs via a bottom-up approach. The present invention encompasses the recognition that, for such applications, controlling the size and shape of cell-laden microgels is important for minimizing diffusion limitations and for exhibiting control over the macroscopic engineered tissue. Micromolding of hydrogels provides a potentially powerful method of fabricating micro- and nano-structures (Khademhosseini et al., 2004, Lab Chip 4:425; and Khademhosseini et al., 2003, Adv. Mater., 15:1995).

### Polymers

In general, cells are encapsulated within a hydrogel by mixing a cell suspension with a precursor solution and crosslinking or polymerizing the resulting mixture. In accordance with the present invention, a precursor solution comprises one or more polymers and/or polymer precursors (e.g., monomers, oligomers, etc.) and, optionally, one or more cells. Any polymer that, upon crosslinking and/or polymerization, is capable of forming a hydrogel can be used in accordance with the present invention.

Polymers to be included in a precursor solution in accordance with the present invention may be natural polymers or unnatural (e.g. synthetic) polymers. In some embodiments, polymers can be linear or branched polymers. In some embodiments, polymers can be dendrimers. Polymers may be homopolymers or copolymers comprising two or more monomers. In terms of sequence, copolymers may be block copolymers, graft copolymers, random copolymers, blends, mixtures, and/or adducts of any of the foregoing and other polymers. Typically, polymers in accordance with the present invention are organic polymers. In some embodiments, polymers may be biocompatible. In some embodiments, polymers may be biodegradable. In some embodiments, polymers may be both biocompatible and biodegradable.

In some embodiments, polymers may be modified with one or more moieties and/or functional groups. Any moiety or functional group can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301).

In general, the percent of polymer in a precursor solution is a percent that allows for the formation of hydrogels in accordance with the present invention. In some embodiments, the percent of polymer in a precursor solution that is suitable for forming hydrogels in accordance with the present invention ranges between about 1% w/w and about 60% w/w, between about 1% w/w and about 50% w/w, between about 1% w/w and about 40% w/w, between about 5% w/w and about 30% w/w, between about 5% w/w and about 20% w/w, or between about 5% w/w and about 10% w/w. In some embodiments, the percent of polymer in a precursor solution that is suitable for forming hydrogels in accordance with the present invention is about 1% w/w, about 2% w/w, about 3% w/w, about 4% w/w, about 5% w/w, about 6% w/w, about 7% w/w, about 8% w/w, about 9% w/w, about 10% w/w, about 20% w/w, about 30% w/w, about 40% w/w, about 50% w/w, about 60% w/w, or more. In some embodiments, the percent of polymer in a precursor solution that is suitable for forming hydrogels in accordance with the present invention is approximately 5% w/w. As a general rule, the absorption capacity of a resulting hydrogel increases with higher polymer concentrations in the precursor solution.

Natural Polymers

In some embodiments, a polymer to be included in a precursor solution in accordance with the present invention may be a natural polymer, such as a carbohydrate, protein, nucleic acid, lipid, etc. In some embodiments, natural polymers may be synthetically manufactured.

Many natural polymers, such as collagen, hyaluronic acid (HA), and fibrin, are derived from various components of the mammalian extracellular matrix. Collagen is one of the main proteins of the mammalian extracellular matrix, while HA is a polysaccharide that is found in nearly all animal tissues. Alginate and agarose are polysaccharides that are derived from marine algae sources. Some advantages of natural polymers include low toxicity and high biocompatibility.

In some embodiments, a polymer to be included in a precursor solution in accordance with the present invention may be a carbohydrate. In some embodiments, a carbohydrate may be a monosaccharide (i.e. simple sugar). In some embodiments, a carbohydrate may be a disaccharide, oligosaccharide, and/or polysaccharide comprising monosaccharides and/or their derivatives connected by glycosidic bonds, as known in the art. Although carbohydrates that are of use in the present invention are typically natural carbohydrates, they may be at least partially-synthetic. In some embodiments, a carbohydrate is a derivatized natural carbohydrate.

In certain embodiments, a carbohydrate is or comprises a monosaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is or comprises a disaccharide, including but not limited to lactose, sucrose, maltose, trehalose, and cellobiose. In certain embodiments, a carbohydrate is or comprises a polysaccharide, including but not limited to hyaluronic acid (HA), alginate, heparin, agarose, chitosan, N,O-carboxylmethylchitosan, chitin, cellulose, microcrystalline cellulose, hydroxypropyl methylcellulose (HPMC), hydroxycellulose (HC), methylcellulose (MC), pullulan, dextran, cyclodextran, glycogen, starch, hydroxyethylstarch, carageenan, glycon, amylose, starch, heparin, konjac, glucommannan, pustulan, curdlan, and xanthan. In certain embodiments, the carbohydrate is a sugar alcohol, including but not limited to mannitol, sorbitol, xylitol, erythritol, maltitol, and lactitol.

In certain embodiments, a polymer to be included in a precursor solution in accordance with the present invention is a glycosaminoglycan, including, but not limited to, hyaluronic acid (HA), chondroitin sulphate, dermatan sulphate, keratan sulphate, and/or heparan sulphate.

In certain specific embodiments, a polymer to be included in a precursor solution in accordance with the present invention is hyaluronic acid (HA), salts thereof, and/or derivatives thereof. HA is a linear polysaccharide composed of β-1,4-linked D-glucuronic acid (GlcUA) and β-1,3 N-acetyl-D-glucosamine (GlcNAc) disaccharide units and is a ubiquitous component of mammalian extracellular matrix. Covalently crosslinked HA hydrogels can be formed by means of multiple chemical modifications (Vercruysse et al., 1997, Bioconjugate Chem., 8:686; Prestwich et al., 1998, J. Controlled Release, 53:93; Burdick et al., 2005, Biomacromolecules, 6:386; and Gamini et al., 2002, Biomaterials, 23:1161). HA is degraded by cells through the release of enzymes such as hyaluronidase.

In certain specific embodiments, a polymer to be included in a precursor solution in accordance with the present invention is alginate, a linear polysaccharide that is derived from brown seaweed and bacteria. It gels under benign conditions, which makes it attractive for cell encapsulation. Alginate gels are formed upon formation of ionic bridges between divalent cations (e.g., Ca²⁺) and various polymer chains of the alginate. The crosslinking density of alginate gels is a function of the monomer units and molecular weight of the polymer. Alginate gels degrade slowly in a process in which the mechanical properties of the gels are altered with time.

In certain specific embodiments, a polymer to be included in a precursor solution in accordance with the present invention is chitosan, which is derived from chitin. Dissolved chitosan can be crosslinked by increasing pH, by dissolving in a nonsolvent (Suh and Matthew, 2000, Biomaterials, 21:2589), or by photocrosslinking (Ishihara et al., 2002, Biomaterials, 23:833). Chitosan can be degraded by the lysosome and is therefore biodegradable (Lee et al., 1995, Biomaterials, 16:1211). Chitosan gels have been used for many applications, including drug delivery (Ruel-Gariepy et al, 2002, J. Controlled Release, 82:373; and Li and Xu, 2002, J. Pharm. Sci., 91:1669). In specific embodiments, a polymer to be included in a precursor solution in accordance with the present invention is N,O-carboxylmethylchitosan.

In certain specific embodiments, a polymer to be included in a precursor solution in accordance with the present invention is heparin.

In certain specific embodiments, a polymer to be included in a precursor solution in accordance with the present invention is agarose.

In certain specific embodiments, a polymer to be included in a precursor solution in accordance with the present invention is dextran.

In certain specific embodiments, a polymer to be included in a precursor solution in accordance with the present invention is cellulose and/or derivatives thereof, including, but not limited to, microcrystalline cellulose, hydroxypropyl methylcellulose (HPMC), hydroxycellulose (HC), methylcellulose (MC).

In some embodiments, a polymer in to be included in a precursor solution in accordance with the present invention may be a protein or peptide. Exemplary proteins that may be used in accordance with the present invention include, but are not limited to, collagen, elastin, fibrin, albumin, poly(amino acids) (e.g. polylysine), glycoproteins, antibodies, etc. In specific embodiments, a polymer to be used in hydrogels in accordance with the present invention is collagen. In specific embodiments, a polymer to be used in hydrogels in accordance with the present invention is elastin. In specific embodiments, a polymer to be used in hydrogels in accordance with the present invention is fibrin.

In certain specific embodiments, a polymer to be included in a precursor solution in accordance with the present invention is collagen. Collagen and other mammalian-derived protein-based polymers can provide effective matrices for cellular growth because they contain many cell-signaling domains present in the in vivo extracellular matrix. Collagen gels can be created through natural means without chemical modifications. To synthesize gels with enhanced mechanical properties, various methods have been developed such as chemical crosslinking (Lee et al., 2001, Biomaterials, 22:3145; and Lee et al, 2003, Tissue Eng., 9:27), crosslinking with UV or temperature (Lee et al., 2001, Biomaterials, 22:3145; and Schoof et al, 2001, J. Biomed. Mater. Res., 58:352), and/or mixing with other polymeric agents (Lee et al., 2001, Biomaterials, 22:3145; and Chen et al., 2000, Key Eng. Mater., 192-1:753). Collagen degradation is mediated through natural means by proteins such as collagenase.

In some embodiments, a peptide sequence can be based on the sequence of a protein. In some embodiments, a peptide sequence can be a random arrangement of amino acids. Proteins may contain L-amino acids, D-amino acids, or both and may contain any of a variety of amino acid modifications or analogs known in the art. Useful modifications include, e.g., terminal acetylation, amidation, lipidation, phosphorylation, glycosylation, acylation, farnesylation, sulfation, etc.

Synthetic Polymers

In some embodiments, polymers to be included in a precursor solution in accordance with the present invention may be synthetic polymers, including, but not limited to, polyethylenes, polycarbonates (e.g. poly(1,3-dioxan-2-one)), polyanhydrides (e.g. poly(sebacic anhydride)), polyhydroxyacids (e.g. poly(β-hydroxyalkanoate)), polypropylfumarates, polycaprolactones, polyamides (e.g. polycaprolactam), polyacetals, polyethers, polyesters (e.g. polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines. In some embodiments, polymers to be included in a precursor solution in accordance with the present invention include polymers which have been approved for use in humans by the U.S. Food and Drug Administration (FDA) under 21 C.F.R. §177.2600, including, but not limited to, polyesters (e.g. polylactic acid, poly(lactic-co-glycolic acid), polycaprolactone, polyvalerolactone, poly(1,3-dioxan-2-one)); polyanhydrides (e.g. poly(sebacic anhydride)); polyethers (e.g., polyethylene glycol); polyurethanes; polymethacrylates; polyacrylates; polycyanoacrylates; copolymers of PEG and poly(ethylene oxide) (PEO).

In some embodiments, polymers to be included in a precursor solution in accordance with the present invention are hydrophilic. For example, polymers may comprise anionic groups (e.g. phosphate group, sulphate group, carboxylate group); cationic groups (e.g. quaternary amine group); or polar groups (e.g. hydroxyl group, thiol group, amine group).

In some embodiments, polymers to be included in a precursor solution in accordance with the present invention are chemically neutral. Neutral synthetic polymers can be generated from derivatives of poly(ethylene glycol) (PEG), poly(hydroxyethyl methacrylate) (PHEMA), and poly(vinyl alcohol) (PVA).

PEG hydrogels are nontoxic, non-immunogenic, inert to most biological molecules (e.g. proteins), and approved by the FDA for various clinical uses. PEG polymers can be covalently crosslinked using a variety of methods to form hydrogels. In some embodiments, PEG chains are crosslinked through photopolymerization using acrylate-terminated PEG monomers (West and Hubbell, 1995, React. Polym., 25:139). In the presence of cells, PEG hydrogels are passive constituents of the cell environment since they prevent adsorption of proteins. However, numerous methods of modifying PEG gels have made PEG gels a versatile template for many subsequent conjugations. For example, peptide sequences have been incorporated into PEG gels to induce degradation (West and Hubbell, 1999, Macromolecules, 32:241) or modify cell adhesion (Hem and Hubbell, 1998, J. Biomed. Mater. Res., 39:266). In addition to chemical modification, block copolymers of PEG, such as triblock copolymers of PEO and poly(propylene oxide) (henceforth designated as PEO-b-PPO-b-PEO), degradable PEO, poly(lactic acid) (PLA), and other similar materials, can be used to add specific properties to the PEG hydrogels (Huh and Bae, 1999, Polymer, 40:6147).

Poly(hydroxyethyl methacrylate) (PHEMA) is characterized by desirable mechanical properties, optical transparency, and stability in water. Like PEG, various modifications can be made to PHEMA derivatives to modify its properties. For example, dextran-modified PHEMA gels have been synthesized to modulate the degradation properties of a hydrogel (Meyvis et al., 2000, Macromolecules, 33:4717). Copolymerization of HEMA monomers with other monomers, such as methyl methacrylate, can be used to modify properties such as swelling and mechanical properties.

PVA hydrogels are stable, elastic gels that can be formed by physical crosslinking methods (e.g. repeated freezing and thawing process), chemical crosslinking methods (e.g. glutaraldehyde, acetaldehyde, formaldehyde, and/or other monoaldehydes), irradiative crosslinking mechanisms (e.g. electron beam and/or gamma irradiation), and/or photo-crosslinking mechanisms (Nuttelman et al., 2001, J. Biomed. Mater., 57:217; Peppas and Merrill, 1977, J. Biomed. Mater. Res., 11:423; Schmedlen et al., 2002, Biomaterials, 23:4325; and Nuttelman et al., 2002, Biomaterials, 23:3617). Physically crosslinked versions of PVA hydrogels are biodegradable, and thus can be used for various biomedical applications (Peppas and Merrill, 1977, J. Biomed. Mater. Res., 11:423; Martens et al., 2003, Biomacromolecules, 4:283; Wan et al., 2002, J. Biomed. Mater. Res., 63:854; Mandal et al., 2002, Pharm. Res., 19:1713; and Shaheen and Yamaura, 2002, J. Controlled Release, 81:367).

Many of the polymers described in the following paragraphs are not necessarily capable of forming hydrogels by themselves. However, these polymers can be used in the preparation of hydrogels as long as the precursor solution and, ultimately, the hydrogel comprises at least one polymer which is capable of forming hydrogels.

In some embodiments, polymers to be included in a precursor solution in accordance with the present invention may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; copolymers of PEG and copolymers of lactide and glycolide (e.g. PLA-PEG copolymers, PGA-PEG copolymers, PLGA-PEG copolymers, and derivatives thereof. In some embodiments, polyesters include, for example, polyanhydrides, poly(ortho ester), poly(ortho ester)-PEG copolymers, poly(caprolactone), poly(caprolactone)-PEG copolymers, polylysine, polylysine-PEG copolymers, poly(ethylene imine), poly(ethylene imine)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer to be included in a precursor solution in accordance with the present invention may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers to be included in a precursor solution in accordance with the present invention may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate)copolymer, polyacrylamide, aminoalkyl methacrylate copolymer, glycidyl methacrylate copolymers, polycyanoacrylates, and combinations comprising one or more of the foregoing polymers. An acrylic polymer may comprise fully-polymerized copolymers of acrylic and methacrylic acid esters with a low content of quaternary ammonium groups.

In some embodiments, polymers to be included in a precursor solution in accordance with the present invention can be cationic polymers. Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine)dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH.

In some embodiments, polymers to be included in a precursor solution in accordance with the present invention can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633).

By tailoring their molecular structure, polymer networks can be created that interact with their environment in a preprogrammed and intelligent manner. Environmentally responsive hydrogels have been synthesized that are capable of sensing and responding to changes to external stimuli, such as changes to pH and temperature (Peppas ed. Hydrogels in Medicine and Pharmacy, CRC, Boca Raton, Fla., 1987; Peppas and Khare, 1993, Adv. Drug Delivery Rev., 11:1; Peppas et al., 2000, Eur. J. Pharm., 50:27; Jeong et al., 2002, Adv. Drug Delivery Rev., 54:37; and Miyata et al., 2002, Adv. Drug Delivery Rev., 54:79).

In some embodiments, the response mechanism is based on the chemical structure of the polymer network (e.g., the functionality of chain side groups, branches, crosslinks, etc.). For example, in networks that contain weakly acidic or basic pendent groups, water adsorption can result in ionization of these pendent groups, depending on the solution pH and ionic composition. The gels then act as semipermeable membranes for the counterions, thereby influencing the osmotic balance between the hydrogel and the external solution through ion exchange, depending on the ion-ion interactions. For ionic gels containing weakly acidic pendent groups, the equilibrium degree of swelling increases as the pH of the external solution increases, while the degree of swelling increases as the pH decreases for gels containing weakly basic pendent groups. Numerous properties (e.g., ionic content, ionization equilibrium considerations, nature of counterions, nature of the polymer, etc.) contribute to the swelling of ionic hydrogels (Khare and Peppas, 1993, J. Biomater. Sci., Polym. Ed., 4:275; Scott and Peppas, 1999, Macromolecules, 63:6149; and Podual and Peppas, 2005, Polym. Int., 54:581). Exemplary ionic polymers include poly(acrylic acid), poly(methacrylic acid), polyacrylamide (PAam), poly(diethylaminoethyl methacrylate), and poly(dimethylaminoethyl methacrylate).

Temperature-responsive hydrogels undergo a reversible volume phase transition with a change in the temperature of the environmental conditions. This type of behavior is related to polymer phase separation as the temperature is raised to a critical value known as the lower critical solution temperature (LCST). Networks showing a lower critical miscibility temperature tend to shrink or collapse as the temperature is increased above the LCST, and the gels swell upon lowering the temperature below the LCST. For example, PNIPAAm exhibits a LCST around 33° C. In some embodiments, temperature-responsive hydrogels are based on poly(N-isopropylacrylamide) (PNIPAAm) and its derivatives (Jeong et al, 2002, Adv. Drug Delivery Rev., 54:37; and Sershen and West, 2003, Adv. Drug Delivery Rev., 55:439).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al, 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

Those skilled in the art will recognize that the polymers listed herein represent an exemplary, not comprehensive, list of polymers that can be included in a precursor solution (and, therefore, eventually in a hydrogel) in accordance with the present invention.

### Cells

In general, cells to be used in accordance with the present invention are any types of cells. In general, the cells should be viable when encapsulated within hydrogels. In some embodiments, cells that can be encapsulated within hydrogels in accordance with the present invention include, but are not limited to, mammalian cells (e.g. human cells, primate cells, mammalian cells, rodent cells, etc.), avian cells, fish cells, insect cells, plant cells, fungal cells, bacterial cells, and hybrid cells. In some embodiments, exemplary cells that can be encapsulated within hydrogels include stem cells, totipotent cells, pluripotent cells, and/or embryonic stem cells. In some embodiments, exemplary cells that can be encapsulated within hydrogels in accordance with the present invention include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc., and/or hybrids thereof, may be encapsulated within hydrogels in accordance with the present invention.

Exemplary mammalian cells that can be encapsulated within hydrogels in accordance with the present invention include, but are not limited to, Chinese hamster ovary (CHO) cells, HeLa cells, Madin-Darby canine kidney (MDCK) cells, baby hamster kidney (BHK cells), NS0 cells, MCF-7 cells, MDA-MB-438 cells, U87 cells, A172 cells, HL60 cells, A549 cells, SP10 cells, DOX cells, DG44 cells, HEK 293 cells, SHSY5Y, Jurkat cells, BCP-1 cells, COS cells, Vero cells, GH3 cells, 9L cells, 3T3 cells, MC3T3 cells, C3H-10T1/2 cells, NIH-3T3 cells, and C6/36 cells.

Exemplary fish cell lines that can be encapsulated in hydrogels in accordance with the present invention include, but are not limited to, ZF4 cells, AB9 cells, GAKS cells, OLF-136 cells, CAEP cells, CAF cells, OLHE-131 cells, OLME-104 cells, ULF-23 cells, BRF41 cells, Hepa-E1 cells, Hepa-T1 cells, GEM-81 cells, GEM-199 cells, GEM-218 cells, GAKS cells, D-11 cells, R1 cells, RTG-2 cells, RTO cells, and TPS cells. A more complete list can be found in Fryer and Lannan, 2005, “Three decades of fish cell culture: a current listing of cell lines derived from fishes,” J. Tissue Culture Methods, 16:87-94.

Any of a variety of cell culture media, including complex media and/or serum-free culture media, that are capable of supporting growth of the one or more cell types or cell lines may be used to grow and/or maintain cells in accordance with the present invention. Typically, a cell culture medium contains a buffer, salts, energy source, amino acids (e.g., natural amino acids, non-natural amino acids, etc.), vitamins, and/or trace elements. Cell culture media may optionally contain a variety of other ingredients, including but not limited to, carbon sources (e.g., natural sugars, non-natural sugars, etc.), cofactors, lipids, sugars, nucleosides, animal-derived components, hydrolysates, hormones, growth factors, surfactants, indicators, minerals, activators of specific enzymes, activators inhibitors of specific enzymes, enzymes, organics, and/or small molecule metabolites. Cell culture media suitable for use in accordance with the present invention are commercially available from a variety of sources, e.g., ATCC (Manassas, Va.). In certain embodiments, one or more of the following media are used to grow cells: RPMI-1640 Medium, Dulbecco's Modified Eagle's Medium, Minimum Essential Medium Eagle, F-12K Medium, Iscove's Modified Dulbecco's Medium.

Those skilled in the art will recognize that the cells listed herein represent an exemplary, not comprehensive, list of cells that can be encapsulated within a precursor solution (and, therefore, eventually in a hydrogel) in accordance with the present invention.

In some embodiments, it is desirable that cells are evenly distributed throughout a hydrogel. Even distribution can help provide more uniform tissue-like hydrogels that provide a more uniform environment for encapsulated cells. In some embodiments, cells are located on the surface of a hydrogel. In some embodiments, cells are located in the interior of a hydrogel. In some embodiments, cells are layered within a hydrogel. In some embodiments, each cell layer within a hydrogel contains different cell types. In some embodiments, cell layers within a hydrogel alternate between two cell types.

In some embodiments, the conditions under which cells are encapsulated within hydrogels are altered in order to maximize cell viability. In some embodiments, for example, cell viability increases with lower polymer concentrations, lower photoinitiator concentration, and shorter UV exposure times. In some embodiments, cells located at the periphery of a hydrogel tend to have decreased viability relative to cells that are fully-encapsulated within the hydrogel. In some embodiments, conditions (e.g. pH, ionic strength, nutrient availability, temperature, oxygen availability, osmolarity, etc.) of the surrounding environment may need to be regulated and/or altered to maximize cell viability.

In some embodiments, cell viability can be measured by monitoring one of many indicators of cell viability. In some embodiments, indicators of cell viability include, but are not limited to, intracellular esterase activity, plasma membrane integrity, metabolic activity, gene expression, and protein expression. To give but one example, when cells are exposed to a fluorogenic esterase substrate (e.g. calcein AM), live cells fluoresce green as a result of intracellular esterase activity that hydrolyzes the esterase substrate to a green fluorescent product. To give another example, when cells are exposed to a fluorescent nucleic acid stain (e.g. ethidium homodimer-1), dead cells fluoresce red because their plasma membranes are compromised and, therefore, permeable to the high-affinity nucleic acid stain.

In general, the percent of cells in a precursor solution is a percent that allows for the formation of hydrogels in accordance with the present invention. In some embodiments, the percent of cells in a precursor solution that is suitable for forming hydrogels in accordance with the present invention ranges between about 0.1% w/w and about 80% w/w, between about 1.0% w/w and about 50% w/w, between about 1.0% w/w and about 40% w/w, between about 1.0% w/w and about 30% w/w, between about 1.0% w/w and about 20% w/w, between about 1.0% w/w and about 10% w/w, between about 5.0% w/w and about 20% w/w, or between about 5.0% w/w and about 10% w/w. In some embodiments, the percent of cells in a precursor solution that is suitable for forming hydrogels in accordance with the present invention is approximately 5% w/w. In some embodiments, the concentration of cells in a precursor solution that is suitable for forming hydrogels in accordance with the invention ranges between about 1×10⁵ cells/ml and 1×10⁸ cells/ml or between about 1×10⁶ cells/ml and 1×10⁷ cells/ml.

In some embodiments, a single hydrogel comprises a population of identical cells and/or cell types. In some embodiments, a single hydrogel comprises a population of cells and/or cell types that are not identical. In some embodiments, a single hydrogel may comprise at least two different types of cells. In some embodiments, a single hydrogel may comprise 3, 4, 5, 10, or more types of cells. To give but one example, in some embodiments, a single hydrogel may comprise only embryonic stem cells. In some embodiments, a single hydrogel may comprise both embryonic stem cells and hematopoietic stem cells.

### Production of Hydrogels

The present invention provides methods for producing cell-laden hydrogels in accordance with the present invention. Inventive cell-laden hydrogels may be manufactured using any available method. To give but one general example, cells can be suspended in a polymer precursor solution and molded using a stamp. Subsequently, the polymer precursor solution can be crosslinked and/or polymerized to form a gel. The mold can then be removed to generate an array of micromolded hydrogels that can be harvested into a solution using a simple wash. A specific application of this general procedure is described in detail in the Exemplification, below.

In some embodiments, inventive methods utilize a stamp in which the precursor solution is molded. In some embodiments, stamps comprise wells and intervening ridges. In some embodiments, each stamp can comprise approximately 5 wells, approximately 10 wells, approximately 50 wells, approximately 100 wells, approximately 500 wells, approximately 1000 wells, or more wells.

In some embodiments, the wells have particular shapes (e.g. square, rectangle, triangle, any polygon, circle, oval, ellipse, cube, cone, sphere, cylinder, tube, plate, disc, etc., or shapes similar to any of the foregoing).

In some embodiments, the wells have a greatest dimension ranging between approximately 0.5 μm and approximately 5000 μm, between approximately 1.0 μm and approximately 1000 μm, between approximately 50 μm and approximately 1000 μm, between approximately 100 μm approximately 1000 μm, between approximately 10 μm and approximately 100 μm, or between approximately 50 μm and approximately 100 μm. In some embodiments, the wells have a greatest dimension of less than approximately 5000 μm, less than approximately 1000 μm, less than approximately 100 μm, less than approximately 10 μm, or smaller. In some embodiments, the wells have a greatest dimension of approximately 1.0 μm, approximately 5 μm, approximately 10 μm, approximately 50 μm, approximately 100 μm, approximately 200 μm, approximately 250 μm, approximately 300 μm, approximately 400 μm, approximately 500 μm, approximately 600 μm, approximately 700 μm, approximately 750 μm, approximately 800 μm, approximately 900 μm, or approximately 1000 μm.

In some embodiments, stamps have a greatest dimension ranging between about 50 μm and several meters. In some embodiments, stamps have a greatest dimension ranging between about 50 μm and about 1000 cm, between about 50 μm and about 100 cm, between about 50 μm and about 10 cm, between about 50 μm and about 1000 mm, between about 50 μm and about 100 mm, between about 50 μm and about 10 mm, between about 50 μm and about 1000 μm, or between about 50 μm and about 100 μm. In some embodiments, stamps can be of a size and shape that is compatible with particular laboratory equipment that is used in the production of hydrogels (e.g. a stamp may be of a shape and size that allows the stamp to be mounted on a standard glass slide).

In some embodiments, stamps are manufactured by curing (e.g. heat-curing) a prepolymer on a master stamp. In some embodiments, a master stamp may comprise a silicone surface, metal surface, plastic surface, ceramic surface, glass surface, etc. After curing, the resulting stamp is peeled off of the master stamp.

In some embodiments, stamps can be made of any material. In general, the surface of the stamp which is exposed to the precursor solution should be hydrophilic. If the stamp is made of a hydrophobic substance, it can be made hydrophilic by any method known in the art (e.g. plasma cleaning, chemical derivitization of surface, etc.). In specific embodiments, the stamp can be made of hydrophilic poly(dimethylsiloxane) (PDMS) and/or PMDS that has been surface-treated such that it is hydrophilic. In specific embodiments, the stamp can be made of Parylene-C. In specific embodiments, the stamp can be made of fluorinated carbon molecules which resemble liquid Teflon®. In some embodiments, it is desirable for stamps to be constructed such that cells are capable of sticking to the well portion of the stamp, but not to the top of the ridges. In specific embodiments, the ridges may comprise a hydrophobic material to which cells do not stick.

After the precursor solution has been introduced into the wells, the stamp can be covered by a coverslip (e.g. a PDMS coverslip). Then the precursor solution is subjected to conditions that induce crosslinking and/or polymerization. Any crosslinking method known in the art can be utilized, including, but not limited to, photocrosslinking, chemical crosslinking mechanisms, physical crosslinking mechanisms, irradiative crosslinking mechanisms, thermal crosslinking mechanisms, ionic crosslinking mechanisms, and the like, described in further detail in the following paragraphs.

In some embodiments, photocrosslinking methods are utilized. Polymers that can be crosslinked using photocrosslinking include, but are not limited to, polysaccharide based hydrogels (e.g. hyaluronic acid, chitosan, dextran, etc.). Photoinitiators produce reactive free radical species that initiate the crosslinking and/or polymerization of monomers upon exposure to light. Any photoinitiator may be used in the crosslinking and/or polymerization reaction. Photoinitiated polymerizations and photoinitiators are discussed in detail in Rabek, Mechanisms of Photophysical Processes and Photochemical Reactions in Polymers, New York: Wiley & Sons, 1987; Fouassier, Photoinitiation, Photopolymerization, and Photocuring, Cincinnati, Ohio: Hanser/Gardner; Fisher et al., 2001, Annu. Rev. Mater. Res., 31:171. A photoinitiator may be designed to produce free radicals at any wavelength of light. In certain embodiments, the photoinitiator is designed to work using UV light (200-500 nm). In certain embodiments, long UV rays are used. In other embodiments, short UV rays are used. In some embodiments, a photoinitiator is designed to work using visible light (400-800 nm). In certain embodiments, a photoinitiator is designed to work using blue light (420-500 nm). In some embodiments, the photoinitiator is designed to work using IR light (800-2500 nm). The output of light can be controlled to provide greater control over the crosslinking and/or polymerization reaction. Control over the reaction in turn results in control over the characteristics and/or properties of the resulting hydrogel. In certain embodiments, the intensity of light ranges from about 500 to about 10,000 μW/cm². In certain embodiments, the intensity of light is about 4000, about 5000, about 6000, about 7000, about 8000, or about 9000 μW/cm². Light may be applied to a precursor solution for about 10 seconds to about 5 minutes. In certain embodiments, light is applied for about 10 to about 60 seconds. In some embodiments, light is applied for about 10 to about 30 seconds. In some embodiments, light is applied for about 20 to about 40 seconds. The light source may allow variation of the wavelength of light and/or the intensity of the light. Light sources useful in the inventive system include, but are not limited to, lamps, fiber optics devices, etc.

In certain embodiments, the photoinitiator is a peroxide (e.g., ROOR′). In certain embodiments, the photoinitiator is a ketone (e.g., RCOR′). In certain embodiments, the compound is an azo compound (e.g., compounds with a —N═N— group). In certain embodiments, the photoinitiator is an acylphosphineoxide. In certain embodiments, the photoinitiator is a sulfur-containing compound. In certain embodiments, the initiator is a quinone. Exemplary photoinitiators include acetophenone; anisoin; anthraquinone; anthraquinone-2-sulfonic acid, sodium salt monohydrate; (benzene) tricarbonylchromium; 4-(boc-aminomethyl)phenyl isothiocyanate; benzin; benzoin; benzoin ethyl ether; benzoin isobutyl ether; benzoin methyl ether; benzoic acid; benzophenone; benzyl dimethyl ketal; benzophenone/1-hydroxycyclohexyl phenyl ketone; 3,3′,4,4′-benzophenonetetracarboxylic dianhydride; 4-benzoylbiphenyl; 2-benzyl-2-(dimethylamino)-4′-morpholinobutyrophenone; 4,4′-bis(diethylamino)benzophenone; 4,4′-bis(dimethylamino)benzophenone; Michler's ketone; camphorquinone; 2-chlorothioxanthen-9-one; 5-dibenzosuberenone; (cumene)cyclopentadienyliron(II) hexafluorophosphate; dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 2,5-dimethylbenzophenone; 3,4-dimethylbenzophenone; diphenyl(2,4,6-trimethylbenzoyl)phosphine oxide; 2-hydroxy-2-methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methybenzoylformate; 2-methyl-4′-(methylthio)-2-morpholinopropiophenone; 9,10-phenanthrenequinone; 4′-phenoxyacetophenone; thioxanthen-9-one; triarylsulfonium hexafluoroantimonate salts; triarylsulfonium hexafluorophosphate salts; 3-mercapto-1-propanol; 11-mercapto-1-undecanol; 1-mercapto-2-propanol; 3-mercapto-2-butanol; hydrogen peroxide; benzoyl peroxide; 4,4′-dimethoxybenzoin; 2,2-dimethoxy-2-phenylacetophenone; dibenzoyl disulphides; diphenyldithiocarbonate; 2,2′-azobisisobutyronitrile (AIBN); camphorquinone (CQ); eosin; dimethylaminobenzoate (DMAB); dimethoxy-2-phenyl-acetophenone (DMPA); Quanta-cure ITX photosensitizer (Biddle Sawyer); Irgacure 907 (Ciba Geigy); Irgacure 651 (Ciba Geigy); Darocur 2959 (Ciba Geigy); ethyl-4-N,N-dimethylaminobenzoate (4EDMAB); 1-[-(4-benzoylphenylsulfanyl)phenyl]-2-methyl-2-(4-methylphenylsulfonyl)propan-1-one; 1-hydroxy-cyclohexyl-phenyl-ketone; 2,4,6-trimethylbenzoyldiphenylphosphine oxide; diphenyl(2,4,6-trimethylbenzoyl)phosphine; 2-ethylhexyl-4-dimethylaminobenzoate; 2-hydroxy-2-methyl-1-phenyl-1-propanone; 65% (oligo[2-hydroxy-2-methyl-1-[4-(1-methylvinyl)phenyl]propanone] and 35% propoxylated glyceryl triacrylate; benzil dimethyl ketal; benzophenone; blend of benzophenone and a-hydroxy-cyclohexyl-phenyl-ketone; blend of Esacure KIP150 and Esacure TZT; blend of Esacure KIP150 and Esacure TZT; blend of Esacure KIP150 and TPGDA; blend of phosphine oxide, Esacure KIP150 and Esacure TZT; difunctional a-hydroxy ketone; ethyl 4-(dimethylamino)benzoate; isopropyl thioxanthone; 2-hydroxy-2-methyl-phenylpropanone; 2,4,6,-trimethylbenzoyldiphenyl phosphine oxide; 2,4,6-trimethyl benzophenone; liquid blend of 4-methylbenzophenone and benzophenone; oligo(2-hydroxy-2 methyl-1-(4(1-methylvinyl)phenyl)propanone; oligo(2-hydroxy-2-methyl-1-4(1-methylvinyl)phenyl propanone and 2-hydroxy-2-methyl-1-phenyl-1-propanone (monomeric); oligo(2-hydroxy-2-methyl-1-4(1-methylvinyl)phenyl propanone and 2-hydroxy-2-methyl-1-phenyl-1-propanone (polymeric); 4-methylbenzophenone; trimethylbenzophenone and methylbenzophenone; and water emulsion of 2,4,6-trimethylbenzoylphosphine oxide, alpha hydroxyketone, trimethylbenzophenone, and 4-methyl benzophenone. In certain embodiments, the photoinitiator is acetophenone; diphenyl(2,4,6-trimethylbenzoyl)phosphine oxide; 4,4′-dimethoxybenzoin; anthraquinone; anthraquinone-2-sulfonic acid; benzene-chromium(0) tricarbonyl; 4-(boc-aminomethyl)phenyl isothiocyanate; benzil; benzoin; benzoin ethyl ether; benzoin isobutyl ether; benzoin methyl ether; benzophenone; benzoic acid; benzophenone/1-hydroxycyclohexyl phenyl ketone, 50/50 blend; benzophenone-3,3′,4,4′-tetracarboxylic dianhydride; 4-benzoylbiphenyl; 2-benzyl-2-(dimethylamino)-4′-morpholinobutyrophenone; 4,4′-bis(diethylamino)benzophenone; Michler's ketone; (±)-camphorquinone; 2-chlorothioxanthen-9-one; 5-dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 3,4-dimethylbenzophenone; diphenyl(2,4,6-trimethylbenzoyl)phosphine oxide/2-hydroxy methylpropiophenone; 4′-ethoxyacetophenone; 2-ethylanthraquinone; ferrocene; 3′-hydroxyacetophenone; 4′-hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methyl benzoylformate; 2-methyl-4′-(methylthio)-2-morpholinopropiophenone; 9,10-phenanthrenequinone; 4′-phenoxyacetophenone; thioxanthen-9-one; triarylsulfonium hexafluorophosphate salts; 3-mercapto-1-propanol; 11-mercapto-1-undecanol; 1-mercapto-2-propanol; and 3-mercapto-2-butanol, all of which are commercially available from Sigma-Aldrich. In certain embodiments, the free radical initiator is selected from the group consisting of benzophenone, benzyl dimethyl ketal, 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1-(4-(1-methylvinyl)phenyl)propanone and 4-methylbenzophenone. In certain embodiments, the photoinitiator is dimethoxy-2-phenyl-acetophenone (DMPA). In certain embodiments, the photoinitiator is a titanocene. In certain specific embodiments, the photoinitiator is 2-hydroxy-1-(4-(hydroxyethoxy)phenyl)-2-methyl-1-propanone. In certain specific embodiments, the photoinitiator is Igracure. In certain embodiments, a combination of photoinitiators is used.

In some embodiments, an initiator of a cationic or anionic crosslinking and/or polymerization process is used. In certain embodiments, the initiator is a photoinitiator of a cationic crosslinking and/or polymerization process. Exemplary photoinitiators of cationic crosslinking and/or polymerization include, but are not limited to, titanium tetrachloride, vanadium tetrachloride, bis(cyclopentadienyl)titanium dichloride, ferrocene, cyclopentadienyl manganese tricarbonyl, manganese decacarbonyl, diazonium salts, diaryliodonium salts (e.g., 3,3′-dinitrodiphenyliodonium hexafluoroarsenate, diphenyliodonium fluoroborate, 4-methoxydiphenyliodonium fluoroborate) and triarylsulfonium salts.

In general, photoinitiators are utilized at concentrations ranging between approximately 0.005% w/w and 5.0% w/w. In some embodiments, photoinitiators are utilized at concentrations of approximately 0.005% w/w, approximately 0.01% w/w, approximately 0.05% w/w, approximately 0.1% w/w, approximately 0.5% w/w, approximately 1.0% w/w, approximately 5.0% w/w, or higher, although high concentrations of photoinitiators may be toxic to cells.

In some embodiments, hydrogel characteristics can be altered and/or controlled by altering photocrosslinking conditions. For example, photocrosslinking utilizing longer wavelengths tends to generate hydrogels with less toxicity. Photocrosslinking for longer periods of time tends to generate hydrogels with stiffer mechanical properties, although higher doses of UV may be toxic to cells. Photocrosslinking utilizing higher-power UV light tends to generate hydrogels with higher mechanical stiffnesses and more extensive crosslinking.

In some embodiments, crosslinking is achieved utilizing chemical crosslinking agents. In some embodiments, chemical crosslinking is achieved using nucleophiles (e.g. amines, thiols, etc.) and/or electrophiles (e.g. acrylates, aldehydes, etc.). Exemplary aldehyde crosslinking agents that can be used in accordance with the present invention include, but are not limited to, glutaraldehyde, acetaldehyde, formaldehyde, and other monoaldehydes. In some embodiments, chemically-based crosslinking methods may include altering the pH such that crosslinking occurs. In some embodiments, chemically-based crosslinking methods may include addition of divalent cations (e.g. Ca²⁺, Mg²⁺, etc.). To give but one example, alginate hydrogels are formed upon formation of ionic bridges between divalent cations and various polymer chains of the alginate.

In some embodiments, crosslinking is achieved utilizing physical crosslinking methods. For example, repeated cycles of freezing and thawing can induce crosslinking of particular polymers.

In some embodiments, crosslinking is achieved utilizing irradiative crosslinking mechanisms. For example, electron beams and/or gamma irradiation can be utilized to induce crosslinks.

In some embodiments, crosslinking is achieved utilizing thermal crosslinking methods. Polymers that can be crosslinked using thermal crosslinking methods include, but are not limited to, agarose and collagen. For example, crosslinks can be induced by the action of a free radical thermal initiator. Any thermal initiator may be used in the crosslinking reaction. In certain embodiments, the thermal initiator is designed to work at a temperature ranging from 30° C. to 120° C. In certain embodiments, the initiator is designed to work at a temperature ranging from 30° C. to 100° C. In other embodiments, the initiator is designed to work at a temperature ranging from 30° C. to 80° C. In certain embodiments, the initiator is designed to work at a temperature ranging from 40° C. to 70° C. In certain particular embodiments, the initiator is designed to work at approximately 30, 40, 50, 60, 70, 80, 90, 100, or 110° C. In certain embodiments, a co-initiator is used. Co-initiators act to lower the decomposition temperature of the initiator. Exemplary co-initiators include, but are not limited to, aromatic amine (e.g., dimethyl aniline), organic peroxides, decahydroacridine 1,8-dione, etc. Heat may be applied to a precursor solution for about 10 seconds to about 5 minutes. In certain embodiments, the heat is applied for about 10 to about 60 seconds. In certain embodiments, the heat is applied for about 10 to about 30 seconds. In certain embodiments, the heat is applied for about 20 to about 40 seconds.

Thermal initiators include peroxides, peracids, peracetates, persulfates, etc. Exemplary thermal initiators include tert-amyl peroxybenzoate; 4,4′-azobis(4-cyanovaleric acid); 1,1′-azobis(cyclohexanecarbonitrile); 2,2′-azobis(2-methylpropionitrile); benzoyl peroxide; 2,2′-azo-bis-isobutyronitrile (AIBN); benzoyl peroxide; 2,2-bis(tert-butylperoxy)butane; 1,1-bis(tert-butylperoxy)cyclohexane; 2,5-bis(tert-butylperoxy)-2,5-dimethylhexane; 2,5-bis(tert-butylperoxy)-2,5-dimethyl-3-hexyne; bis[1-(tert-butylperoxy)-1-methylethyl]benzene; 1,1-bis(tert-butylperoxy)-3,3,5-trimethylcyclohexane; tert-butyl hydroperoxide; tert-butyl peracetate; tert-butyl peracetic acid; tert-butyl peroxide; tert-butyl peroxybenzoate; tert-butylperoxy isopropyl carbonate; cumene hydroperoxide; cyclohexanone peroxide; dicumyl peroxide; lauroyl peroxide; 2,4-pentanedione peroxide; peracetic acid; and potassium persulfate. Many of the above listed thermal initiators are available from commercial sources such as Sigma-Aldrich. In certain embodiments, the initiator is 2,2′-azo-bis-isobutyronitrile (AIBN). In other embodiments, the initiator is benzoyl peroxide (also known as dibenzoyl peroxide). In certain embodiments, a combination of thermal initiators is used. In certain embodiments, the polymerization initiator is a combination of ammonium persulfate (APS) and N,N,N′,N′-tetramethylethylenediamine (TEMED).

In some embodiments, crosslinking is achieved utilizing ionic crosslinking methods. For example, ionic crosslinking methods may be based upon the interaction between cations (e.g. Na⁺, Ca²⁺, Mg²⁺, etc.) and negatively charged functional groups (e.g. carboxylic acids).

After hydrogels are formed, they are removed from the stamps. In some embodiments, hydrogels slide out of the stamps without any need for mechanical force. In some embodiments, hydrogels do not slide out of the stamps by themselves and require mechanical force to be removed from the stamps.

In some embodiments, microfluidics can be used to fabricate hydrogels. For example, the precursor solution can be flowed into a microfluidic channel and a mask can be placed on the microchannel such that cell-laden hydrogels can be fabricated.

In some embodiments, hydrogels in accordance with the present invention may comprise one or more solvents. In general, solvents to be used with hydrogels in accordance with the present invention are water miscible. Exemplary water-miscible solvents to be used in accordance with the present invention include, but are not limited to, alcohols (e.g. methanol, ethanol, isopropanol, butanol, etc.), dimethyl sulfoxide (DMSO), dimethyl formamide (DMF), etc. In certain embodiments, solvents to be used with hydrogels in accordance with the present invention are aqueous. In certain embodiments, solvents to be used with hydrogels in accordance with the present invention are not aqueous.

### Hydrogel Assemblies

In some embodiments, inventive cell-laden microscale hydrogels (i.e. microgels) can be assembled into hydrogel assemblies comprising a plurality of individual microgels. Among other things, such hydrogel assemblies can be useful for forming tissues and/or organs for medical therapies, as described in further detail below in the section entitled “Therapeutic Applications.”

In general, microgels are large enough to encapsulate at least one cell. In some embodiments, microgels have a greatest dimension ranging between approximately 0.5 μm and approximately 5000 μm, between approximately 1.0 μm and approximately 1000 μm, between approximately 50 μm and approximately 1000 μm, between approximately 100 μm approximately 1000 μm, between approximately 10 μm and approximately 100 μm, or between approximately 50 μm and approximately 100 μm. Microgels typically have a greatest dimension of less than approximately 5000 μm. In some embodiments, microgels have a greatest dimension of less than approximately 5000 μm, less than approximately 1000 μm, less than approximately 100 μm, less than approximately 10 μm, or smaller. In some embodiments, microgels have a greatest dimension of approximately 1.0 μm, approximately 5 μm, approximately 10 μm, approximately 50 μm, approximately 100 μm, approximately 200 μm, approximately 250 μm, approximately 300 μm, approximately 400 μm, approximately 500 μm, approximately 600 μm, approximately 700 μm, approximately 750 μm, approximately 800 μm, approximately 900 μm, or approximately 1000 μm.

In some embodiments, microgels stack together in a linear fashion to form chain-like hydrogel assemblies. In some embodiments, microgels stack together in a two-dimensional fashion to form sheet-like hydrogel assemblies. In some embodiments, microgels stack together in a three-dimensional fashion to form cuboid, rectangular, spherical, conical, pyramid-like, cylindrical, tubular, ring-shaped, tetrahedral, hexagonal, octagonal, other regularly-shaped, and/or irregularly-shaped hydrogel assemblies. Although there are numerous possibilities for hydrogel assembly shape, a few exemplary hydrogel assemblies are described in the following paragraphs. These examples are not meant to be limiting, but instead are set forth to provide a flavor of the types of structures that inventive hydrogel assemblies may possess. One of ordinary skill in the art reading the specification will understand that individual microgels of various shapes and sizes can be assembled in numerous ways to generate hydrogel assemblies having desired shapes and structures.

In some embodiments, hydrogel assemblies are tubular, comprising individual ring-shaped microgels which are stacked together to form a tube-like structure. The ring-shaped microgels are generally circular in shape and typically contain a void near the center of the microgel in which microgel substance is not present. The ring-shaped microgels may or may not be identical to one another. In some embodiments, the hydrogel assembly comprises a linear arrangement of two different types of ring-shaped microgels (e.g. wherein one type of microgel encapsulates one cell type, and a second type of microgel encapsulates a second cell type) in an alternating pattern. The ring-shaped microgels may or may not be identical to one another. In some embodiments, the hydrogel assembly comprises a linear arrangement of three different types of ring-shaped microgels in an alternating pattern.

In some embodiments, hydrogel assemblies are spherical, comprising individual cone-shaped microgels. The cone-shaped microgels may or may not be identical to one another. In some embodiments, the hydrogel assembly comprises an arrangement of two different types of ring-shaped microgels (e.g. wherein one type of microgel encapsulates one cell type, and a second type of microgel encapsulates a second cell type). In some embodiments, the hydrogel assembly comprises an arrangement of three or more different types of ring-shaped microgels in an alternating pattern.

In some embodiments, hydrogel assemblies are rectangular, comprising individual square-shaped microgels. The square-shaped microgels may or may not be identical to one another. In some embodiments, the hydrogel assembly comprises an arrangement of two different types of square-shaped microgels (e.g. wherein one type of microgel encapsulates one cell type, and a second type of microgel encapsulates a second cell type). In some embodiments, the hydrogel assembly comprises an arrangement of three or more different types of square-shaped microgels in an alternating pattern.

In some embodiments, such as in the three examples just described, hydrogel assemblies may comprise microgels that all have the same shape as one another. In other embodiments, hydrogel assemblies may comprise microgels that do not all have the same shape as one another. To give but one example, hydrogel assemblies may comprise square-shaped microgels as well as triangular-shaped microgels.

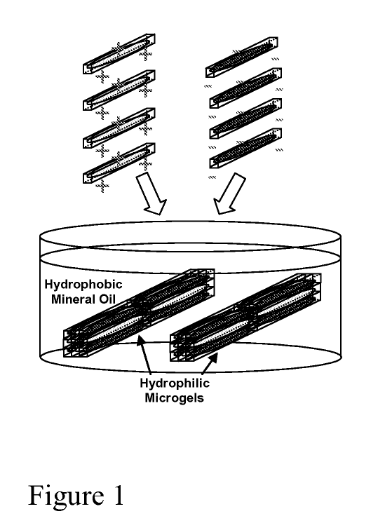

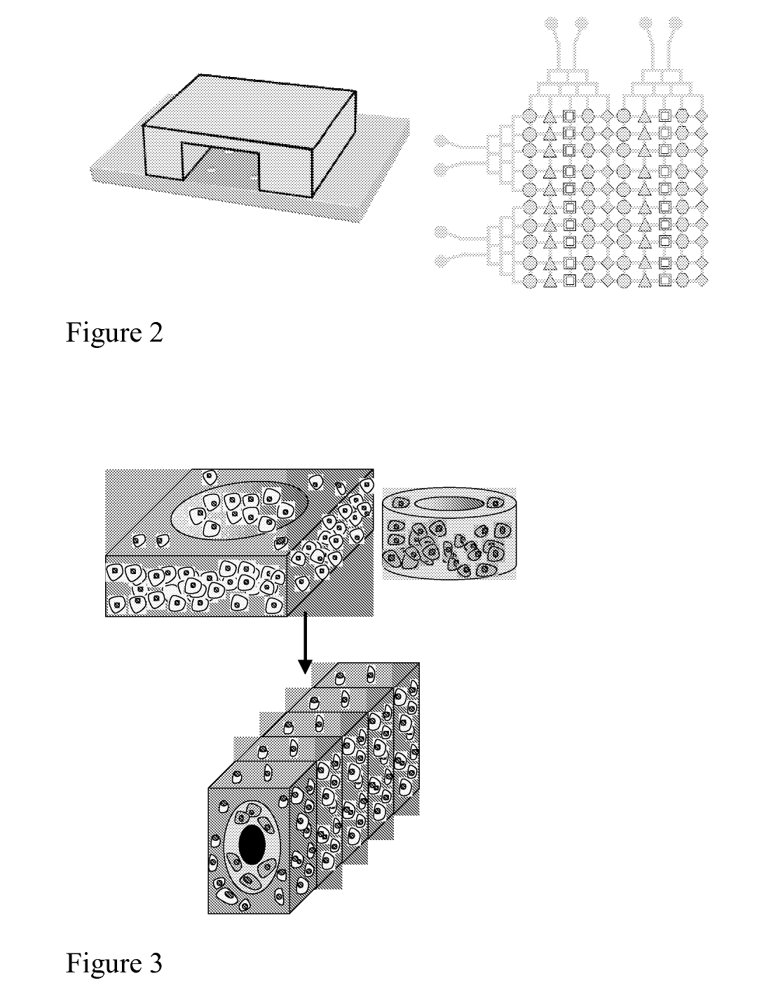

Hydrogel assemblies in accordance with the present invention can be formed using any available method. In some embodiments, hydrogel assemblies are formed by self-assembly (FIG. 1). In specific embodiments, surface modification of hydrogels can be used as a method of initiating self-assembly. For example, by modifying specific surfaces of a hydrogel, it is possible to induce specific interactions such as hydrophobic forces, electrostatic forces, polymer chain entanglement, and/or affinity interactions (e.g. ligand/receptor interactions, antibody/antigen interactions, etc.) for initiating self assembly. Functional groups (e.g. carboxyl groups, amino groups, hydroxyl groups, etc.) may be incorporated into inventive hydrogels to provide chemical moieties that are useful for surface modification. In some embodiments, functional groups are incorporated into inventive hydrogels to provide a negatively- or positively-charged surface of the hydrogel. In some embodiments, hydrogel surfaces can be modified with moieties that can participate in affinity interactions, such as proteins (e.g. antibody/antigen interactions, ligand/receptor interactions, streptavidin/biotin interactions), nucleic acids (e.g. aptamer/target interactions), metals (e.g. nickel/polyhistidine interactions), carbohydrates, lipids, small molecules, drugs, or other adhesive ligands. Hydrogel surfaces can be modified with any substances that specifically bind to one another with high affinity such that individual microgels bind to one another via affinity interactions. High throughput modification of these micromaterials may be achieved by placing a grid of microfluidic channels overtop the micromolded materials such that various solutions and/or moieties can be delivered to the hydrogels (FIG. 2).

Alternatively to surface modification or additionally, microgel shape can be controlled so that the microgels can “self-assemble” into tissues using geometric lock-and-key mechanisms (FIG. 3).

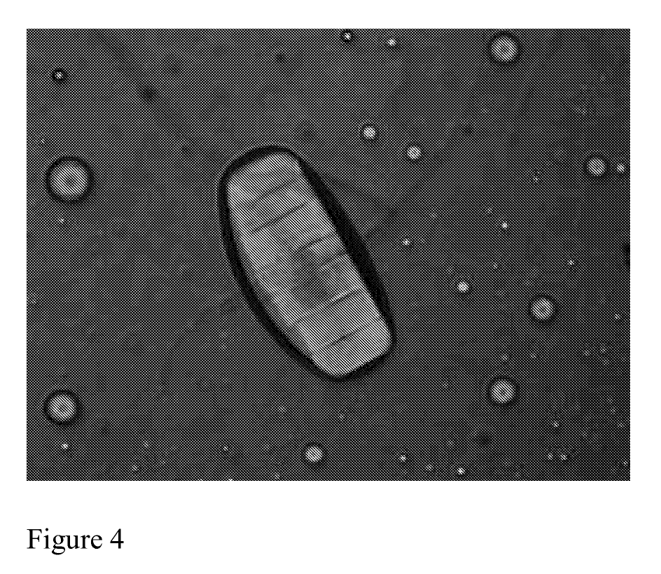

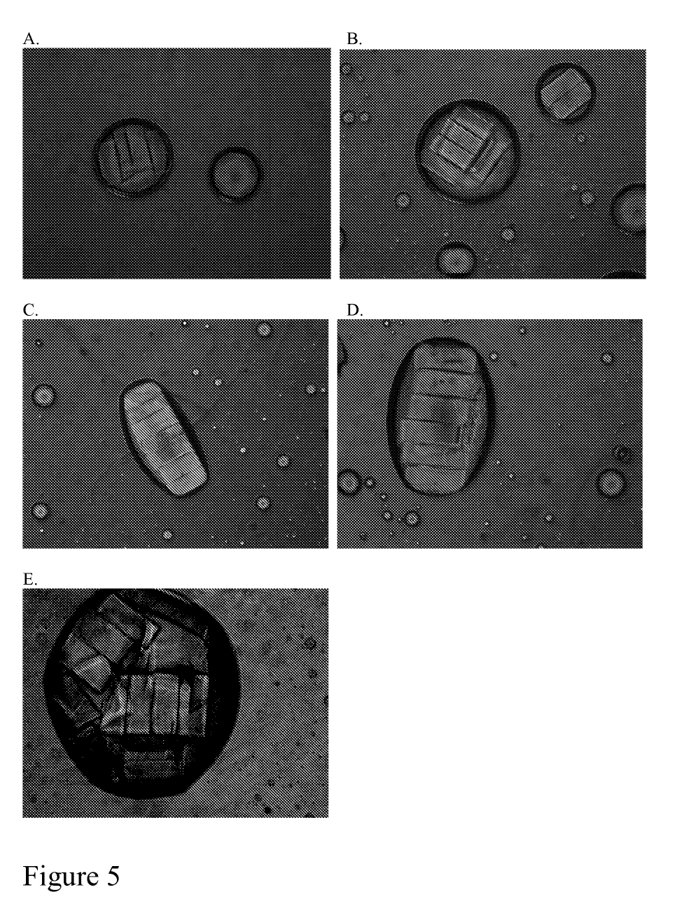

In some embodiments, microgels may be assembled into hydrogel assemblies using surface tension mechanisms. In this approach, individual cell-laden microgels are placed within a hydrophobic bath comprising oil (e.g. almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof) and/or water-immiscible organic solvent (e.g. carbon tetrachloride, chloroform, pentane, hexane, heptane, ether, diethyl ether, toluene, etc.). When aqueous microgels are suspended in oil, a two-phase system is developed, and each microgel is separated from the oil phase by a surface tension. Based on energies, surface tension is typically minimized when the microgels merge, forming assemblies and/or aggregates. Thus, by engineering microgel shapes and sizes, it is possible to induce their assembly in a controlled manner. FIG. 4 shows seven microgels that have assembled in a hydrophobic solution to form a hydrogel assembly. FIG. 5 shows additional examples of hydrogel assemblies at various time points during formation.

In some embodiments, a micromanipulator can be utilized to assemble microgels into hydrogel assemblies. For example, using a micromanipulator, HA microgels and uncrosslinked HA (i.e. glue) can be assembled manually. The unreacted acrylate groups will allow for crosslinking of adjacent microgels.

Once hydrogels assemblies are formed, it is possible to further induce their crosslinking to coalesce the individual microgels to one another. This can be accomplished using a multistep crosslinking approach in which the gels are partially crosslinked, assembled into hydrogel assemblies, and further crosslinked.

In some embodiments, all of the individual microgels of the plurality of microgels are of identical composition, shape, size, etc. In some embodiments, some of the microgels of the plurality of microgels have one particular composition, shape, size, etc., and others of the microgels of the plurality of microgels have another particular composition, shape, size, etc. To give but one example, some of the microgels of the plurality of microgels comprise one particular cell type and others of the microgels of the plurality of microgels comprise a different cell type. Each microgel can contain an individual cell type and through assembly with other microgels that contain different cell types, complex tissues with controlled architecture and spatial distribution of cells can be generated. To give another example, some of the microgels of the plurality of microgels may be rectangular in shape and others of the microgels of the plurality of microgels may be triangular in shape.

### Therapeutic Applications

Hydrogels, hydrogel assemblies, and/or methods described herein can be used for the treatment and/or diagnosis of any disease, disorder, and/or condition which is associated with a tissue specific and/or cell type specific marker. Subjects include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Tissue Engineering

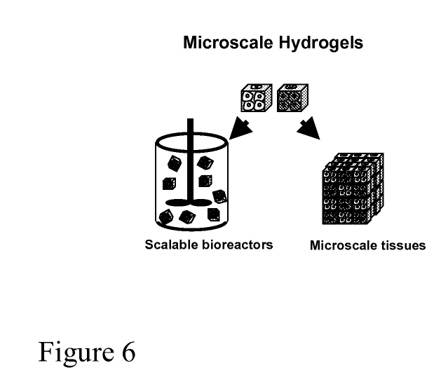

Cell-laden hydrogels and/or hydrogel assemblies in accordance with the present invention may be used for tissue engineering applications (see, for example, FIG. 6). In some embodiments, tissue engineering aims to replace, repair, and/or regenerate tissue and/or organ function or to create artificial tissues and organs for transplantation (Langer and Vacanti, 1993, Science, 260:920). In general, scaffolds used in tissue engineering (e.g. hydrogel scaffolds) mimic the natural extracellular matrix (ECM) and provide support for cell adhesion, migration, and proliferation. Ideally, they allow for differentiated function, new tissue generation, and its 3D organization. Desired characteristics of hydrogel scaffolds include physical parameters such as mechanical strength and degradability, while biological properties include biocompatibility and the ability to provide a biologically relevant microenvironment. Biodegradable hydrogels are advantageous because after tissue is grown, the resulting structures are made entirely or almost entirely from biological components.

In some embodiments, hydrogels and/or hydrogel assemblies can be used for many tissue-engineering applications, including growth of bone (Burdick and Anseth, 2002, Biomaterials, 23:4315; Burdick et al., 2003, Biomaterials, 24:1613; and Burdick et al., 2002, J. Controlled Release, 83:53), cartilage (Bryant et al., 2004, Biotechnol. Bioeng., 86:747; Bryant and Anseth, 2003, J. Biomed. Mater. Res., Part A, 64:70; and Elisseeff et al., 2000, J. Biomed. Mater. Res., 51:164), vascular tissues (Mann et al., 2001, Biomaterials, 22:3045), cardiac tissues, endocrine glands, liver, renal tissue, lymph nodes, pancreas, and other tissues (Hem and Hubbell, 1998, J. Biomed. Mater. Res., 39:266). In some embodiments, hydrogels and/or hydrogel assemblies can be used to deliver signals to cells, act as support structures for cell growth and function, and provide space filling (Lee and Mooney, 2001, Chem. Rev., 101:1869; Rowley et al., 1999, Biomaterials, 20:45; Elisseeff et al., 2001, J. Orthop. Res., 19:1098; and Anseth and Burdick, 2002, MRS Bull., 27:130).

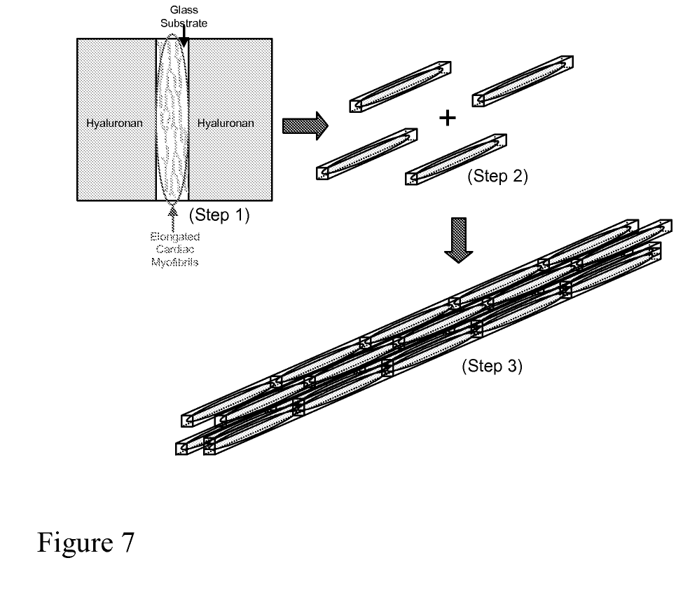

In specific embodiments, tissue engineering can be utilized to provide a potential method of generating a sufficient supply of cardiac tissues for transplantation (Khademhosseini et al., 2006, Proc. Natl. Acad. Sci., USA, 103:2480; and Langer and Vacanti, 1993, Science, 260:920). Existing tissue engineering approaches that involve culturing cells on porous 3D scaffolds do not provide appropriate architecture and function of engineered tissues, making them unsuitable for engineering of complex organs such as the heart. For example, in contrast to native myocardium which comprises elongated cells fused as oriented myofibrils that form contractile fibers, cells within 3D scaffolds generate tissues with randomly oriented cell alignments. This lack of the proper orientation provides a technical obstacle in generating functional cardiac cells. In addition, the relative orientation of cardiac fibers cannot be organized relative to each other. Therefore, the present invention provides methods of producing cell-laden hydrogels in which proper microscale tissue properties (e.g. alignment of cells and the 3D orientation of cardiac fibers) are engineered (FIG. 7).

In some embodiments, hydrogels and/or hydrogel assemblies in accordance with the present invention to be used for tissue engineering applications can be formed in situ, enabling the polymer to conform to the shape of the implantation site. In situ hydrogel formation can be accomplished using methods to achieve crosslinking that can be performed remotely (e.g. photocrosslinking, temperature-based crosslinking, etc.). In such embodiments, a precursor solution comprising cells and at least one polymeric component can be delivered to a target site by injection, for example. After delivery, light of an appropriate wavelength and duration can be applied to the precursor solution, resulting in crosslinking of the polymeric matrix and in situ formation of a cell-laden hydrogel which is tailored to the shape of the target site.

In some embodiments, a plurality of microscale hydrogels (i.e. microgels) can be assembled together to form hydrogel assemblies that are useful for tissue engineering applications (e.g. formation of tissues, organs, etc.). Any method can be used for producing hydrogel assemblies, including, but not limited to, the methods described above in the section entitled “Hydrogel Assemblies.” Utilizing such methods, tissues and/or organs (e.g. bone, cartilage, vascular tissues, cardiac tissues, endocrine glands, liver, renal tissue, lymph nodes, pancreas) can be engineered in which cells are encapsulated within microscale materials (e.g. biodegradable hydrogels). Each of a plurality of microgels can contain an individual cell type and, through assembly with other microgels that contain different cell types (e.g. ordered stacking, self-assembly, etc.), complex tissues with controlled architecture and spatial distribution of cells can be generated.

Drug Delivery

In some embodiments, methods described herein may be used to construct complex delivery devices capable of precisely defined release profiles. This could be achieved through combining drugs or drug delivery devices (i.e. nanoparticles or microparticles) with inventive cell-laden hydrogels and/or hydrogel assemblies and using these to construct more complex drug delivery systems. To give but one example, inventive cell-laden hydrogels may additionally comprise a therapeutic agent to be delivered (e.g. a small molecule, nucleic acid, protein, lipid, and/or carbohydrate drug). Such hydrogels may be useful for delivering a drug to a site that has been targeted for tissue regeneration. For example, a hydrogel comprising osteoinductive cells which is administered to a subject for purposes of regenerating new bone may additionally comprise one or more bone morphogenetic proteins (BMPs) which, upon their release, can help further stimulate the growth of new bone.

In some embodiments, cell-laden hydrogels and/or hydrogel assemblies in accordance with the invention to be utilized for drug delivery can be altered in ways that result in enhanced residence times, sustained drug delivery, and/or targeted drug delivery. Hydrogel properties, such as permeability (e.g., sustained-release applications), enviro-responsive nature (e.g., pulsatile-release applications), surface functionality (e.g., PEG coatings for stealth release), biodegradability (e.g., bioresorbable applications), and surface biorecognition sites (e.g., targeted release and bioadhesion applications), can be altered and/or optimized for controlled drug-delivery applications. For example, by controlling polymer chain length, polymer composition, and/or polymer concentration, it is possible to control the density and degree of crosslinking within a hydrogel. Control over the density and degree of crosslinking provides, among other things, control over sustained-release properties of the resulting hydrogel.

In some embodiments, temperature- and/or pH-responsive hydrogels and/or hydrogel assemblies in accordance with the invention can be used to alter and/or control the properties of hydrogels used for drug delivery. In some embodiments, enzymes can be encapsulated within environmentally responsive hydrogels to create drug delivery systems that are responsive to biological analytes.

In Vitro Tissue Culture

In some embodiments, cell-laden hydrogels and/or hydrogel assemblies in accordance with the invention can be utilized for in vitro tissue culture applications. In certain embodiments, cell-laden hydrogels and/or hydrogel assemblies in accordance with the invention can be utilized to develop assays that are useful for drug discovery and biological studies (e.g. assemble arrays of well-defined constructs for high-throughput drug screening). For example, the presence of feeder cells (e.g. endothelial cells or fibroblasts) in the presence of functional cells (e.g. hepatocytes) can be used to increase the maintenance of the functional cell type. Thus, it is possible to generate 3D structures that mimic the native structure of functional organs that can be subsequently used for drug discovery and/or diagnostics assays.

In some embodiments, cell-laden hydrogels and/or hydrogel assemblies in accordance with the invention can be utilized for toxicity assays that can test the toxicity of a test substance (e.g. utilizing hydrogels in which liver cells have been encapsulated).

Microencapsulating Cells for Immunoisolation

In some embodiments, cell-laden hydrogels and/or hydrogel assemblies in accordance with the invention can be used to encapsulate cells within hydrogels in order to protect the cells from the immune system upon implantation into a subject. Thus, the hydrogel can act as a barrier that prevents immune cells and/or antibodies from destroying the cells contained within the hydrogel.

Microfluidic Channels

In some embodiments, cell-laden hydrogels and/or hydrogel assemblies in accordance with the invention can be used to make various structures, such as microfluidic channels. In this approach, the walls of the microchannels are be made from hydrogel assemblies instead of from more commonly-used materials such as glass and PDMS. Microfluidic channels made from hydrogel assemblies could be useful for many purposes, for example, in applications wherein it is desirable for the walls of the microfluidic channel to be porous.

Diagnostics

In some embodiments, cell-laden hydrogels and/or hydrogel assemblies in accordance with the invention can be used diagnostic applications. To give but one example, cell laden hydrogels can be used for generating tissue-like hydrogels and/or hydrogel assemblies that can be used in assays which test for the presence of one or more particular microbes. For example, if a microbe (e.g. bacteria, viruses, fungi, etc.) were known to specifically bind to a particular tissue, then tissue-like hydrogels and/or hydrogel assemblies could be fabricated that would test for the presence of the microbe in the sample.

Pharmaceutical Compositions

The present invention provides precursor solutions comprising cells and at least one polymer; and one or more pharmaceutically acceptable excipients. The present invention provides novel cell-laden hydrogels comprising cells and at least one polymer; and one or more pharmaceutically acceptable excipients. The present invention provides novel cell-laden hydrogel assemblies comprising cells and at least one polymer; and one or more pharmaceutically acceptable excipients. In some embodiments, the present invention provides for pharmaceutical compositions comprising inventive cell-laden hydrogels as described herein. Such pharmaceutical compositions may optionally comprise one or more additional therapeutically-active substances. In accordance with some embodiments, a method of administering a pharmaceutical composition comprising inventive compositions to a subject in need thereof is provided. In some embodiments, inventive compositions are administered to humans. For the purposes of the present invention, the phrase “active ingredient” generally refers to an inventive cell-laden hydrogel comprising cells and at least one polymer.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions of the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

Pharmaceutical formulations of the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by the FDA. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

General considerations in the formulation and/or manufacture of pharmaceutical agents may be found, for example, in Remington: The Science and Practice of Pharmacy 21^{st} ed., Lippincott Williams & Wilkins, 2005.

Administration

In some embodiments, a therapeutically effective amount of an inventive cell-laden hydrogel and/or hydrogel assembly is delivered to a patient and/or organism prior to, simultaneously with, and/or after diagnosis with a disease, disorder, and/or condition. In some embodiments, a therapeutic amount of an inventive cell-laden hydrogel and/or hydrogel assembly is delivered to a patient and/or organism prior to, simultaneously with, and/or after onset of symptoms of a disease, disorder, and/or condition. In some embodiments, the amount of inventive cell-laden hydrogel and/or hydrogel assembly is sufficient to treat, alleviate, ameliorate, relieve, delay onset of, inhibit progression of, reduce severity of, and/or reduce incidence of one or more symptoms or features of the disease, disorder, and/or condition.

Cell-laden hydrogels, hydrogel assemblies, and/or precursor solutions for in situ hydrogel formation, according to the method of the present invention, may be administered using any amount and any route of administration effective for treatment. The exact amount required will vary from subject to subject, depending on the species, age, and general condition of the subject, the severity of the infection, the particular hydrogel, its mode of administration, its mode of activity, and the like. Compositions in accordance with the invention are typically formulated in dosage unit form for ease of administration and uniformity of dosage. It will be understood, however, that the total daily usage of the hydrogels and/or hydrogel assemblies of the present invention will be decided by the attending physician within the scope of sound medical judgment. The specific therapeutically effective dose level for any particular subject or organism will depend upon a variety of factors including the disorder being treated and the severity of the disorder; the activity of the specific active ingredient employed; the specific polymer and/or cells employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

Cell-laden hydrogels, hydrogel assemblies, and/or precursor solution for in situ hydrogel formation may be administered by any route. In some embodiments, compositions of the present invention are administered by a variety of routes, including direct administration to an affected site. For example, inventive compositions may be administered locally near a site which is in need of tissue regeneration. Local administration may be achieved via injection of hydrogel precursor solution directly to a site in need of tissue regrowth followed by crosslinking, such that a cell-laden hydrogel is formed in situ.

In general, the most appropriate route of administration will depend upon a variety of factors including the identity of the composition to be delivered (e.g. delivery of a precursor solution versus delivery of a hydrogel or hydrogel assembly), nature of the agent (e.g., stability of hydrogel at the site of implantation), the condition of the subject (e.g., whether the subject is able to tolerate the procedure of hydrogel implantation), etc. The invention encompasses the delivery of the inventive cell-laden hydrogel by any appropriate route taking into consideration likely advances in the sciences of drug delivery.

In certain embodiments, cell-laden hydrogels of the invention may be administered such that encapsulated cells and/or therapeutic agents to be delivered are released at concentrations ranging from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

In some embodiments, the present invention encompasses “therapeutic cocktails” comprising inventive cell-laden hydrogels and/or hydrogel assemblies. In some embodiments, cell-laden hydrogels and/or hydrogel assemblies comprise a single cell type and, optionally, a therapeutic agent. In some embodiments, cell-laden hydrogels and/or hydrogel assemblies comprise multiple different cell types and, optionally, a therapeutic agent.

It will be appreciated that cell-laden hydrogels in accordance with the present invention can be employed in combination therapies. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, an inventive hydrogel comprising a certain cell type to be used to promote tissue growth may be administered concurrently with another therapeutic agent used to stimulate growth of the same tissue), or they may achieve different effects (e.g., control of any adverse effects, such as inflammation, infection, etc.).

### Kits

The invention provides a variety of kits comprising one or more of the hydrogels and/or hydrogel assemblies of the invention. For example, the invention provides a kit comprising an inventive hydrogel and/or hydrogel assembly and instructions for use. A kit may comprise multiple different hydrogels and/or hydrogel assemblies. A kit may optionally comprise polymers, precursor solutions, cells, crosslinking agents, etc. A kit may comprise any of a number of additional components or reagents in any combination. All of the various combinations are not set forth explicitly but each combination is included in the scope of the invention. A few exemplary kits that are provided in accordance with the present invention are described in the following paragraphs.

According to certain embodiments of the invention, a kit may include, for example, (i) a precursor solution comprising a cell, a polymer, and a crosslinking initiator; and (ii) instructions for forming a hydrogel from the precursor solution.

In some embodiments, a kit may include, for example, (i) a precursor solution comprising a cell, a polymer, and a crosslinking initiator; and (ii) instructions for administering the precursor solution to a patient in need thereof and performing a crosslinking step such that a cell-laden hydrogel is formed in situ.

In some embodiments, a kit may include, for example, (i) a plurality of microgels, each comprising a cell and at least one polymer; and (ii) instructions for forming a hydrogel assembly from the plurality of microgels.

In some embodiments, a kit may include, for example, (i) a plurality of microgels, each comprising a cell and at least one polymer; and (ii) instructions for forming a hydrogel assembly from the plurality of microgels and for administering the hydrogel assembly to a subject in need thereof.

Kits typically include instructions for use of inventive cell-laden hydrogels. Instructions may, for example, comprise protocols and/or describe conditions for production of hydrogels, administration of hydrogels to a subject in need thereof, production of hydrogel assemblies, etc. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed.

### EXEMPLIFICATION

### Example 1

### Micromolding of Shape-Controlled, Harvestable Cell-Laden Hydrogels

Encapsulation of mammalian cells within hydrogels has great utility for a variety of applications ranging from tissue engineering to cell-based assays. This example presents a technique to encapsulate live cells in three-dimensional (3D) microscale hydrogels (i.e. “microgels”) of controlled shapes and sizes in the form of harvestable free standing units. Cells were suspended in methacrylated hyaluronic acid (MeHA) or poly(ethylene glycol)diacrylate (PEGDA) hydrogel precursor solution containing photoinitiator, micromolded using a hydrophilic poly(dimethylsiloxane) (PDMS) stamp, and crosslinked using ultraviolet (UV) radiation. By controlling the features on the PDMS stamp, the size and shape of the molded hydrogels were controlled. Cells within microgels were well distributed and remained viable. These shape-specific microgels could be easily retrieved, cultured, and potentially assembled to generate structures with controlled spatial distribution of multiple cell types. Further development of this technique may lead to applications in 3D co-cultures for tissue/organ regeneration and cell-based assays in which it is important to mimic the architectural intricacies of physiological cell-cell interactions.

### Materials and Methods

Cell Culture

All cells were manipulated under sterile tissue culture hoods and maintained in a 95% air/5% CO₂ humidified incubator at 37° C. NIH-3T3 mouse embryonic fibroblast cells were maintained in Dulbecco's modified Eagle media (DMEM) supplemented with 10% FBS. Confluent dishes of NIH-3T3 cells were passaged and fed every 3-4 days. Murine embryonic stem (ES) cells (R1 strain) were maintained on gelatin treated dishes with media comprised of 15% ES qualified FBS in DMEM knockout medium. ES cells, were fed daily and passaged every 3 days at a subculture ratio of 1:4.

Prepolymer Solution

Two macromers were used: poly(ethylene glycol) (PEG) and hyaluronic acid (HA). The synthesis of methacrylated HA (MeHA) was previously described (Smeds et al., 2001, J. Biomed. Mater. Res., 54:115). Briefly, the synthesis was performed by the addition of 1 wt % methacrylic anhydride (Sigma) to a solution of 1 wt % HA (Lifecore, MW=67 kDa) in deionized water. The reaction was performed for 24 hours on ice and maintained at a pH of 8-9 through the addition of 5 N NaOH. The macromer solution was then purified by dialyzing (Pierce Biotechnology, MW cutoff 7 kDa) for 48 hours in deionized water and lyophilized for 3 days, resulting in a final dry form which was frozen for storage. The prepolymer form of MeHA was created by dissolving dry MeHA in PBS (Gibco) at 37° C. for 24 hours to facilitate full dissolution. Immediately prior to UV photopolymerization, varying concentrations of photoinitiator solution were added to the prepared prepolymer solution. The photoinitiator solution was 33 wt % 2-hydroxy-1-(4-(hydroxyethoxy)phenyl)-2-methyl-1-propanone (Irgacure 2959, CIBA Chemicals) in methanol.

To generate PEG hydrogels, a solution containing 10% (w/w) poly(ethylene glycol)-diacrylate polymer, PEGDA, (MW 575, Sigma) in phosphate-buffered saline (PBS, Gibco) was prepared prior to experiments in order to allow the PEGDA to adequately dissolve into solution. Immediately prior to UV photopolymerization, photoinitiator solution was added to the prepolymer solution at 1 wt %. The photoinitiator solution was 33% (w/w) 2-hydroxy-1-(4-(hydroxyethoxy)phenyl)-2-methyl-1-propanone (Irgacure 2959, CIBA Chemicals) in methanol.

PDMS Mold Fabrication

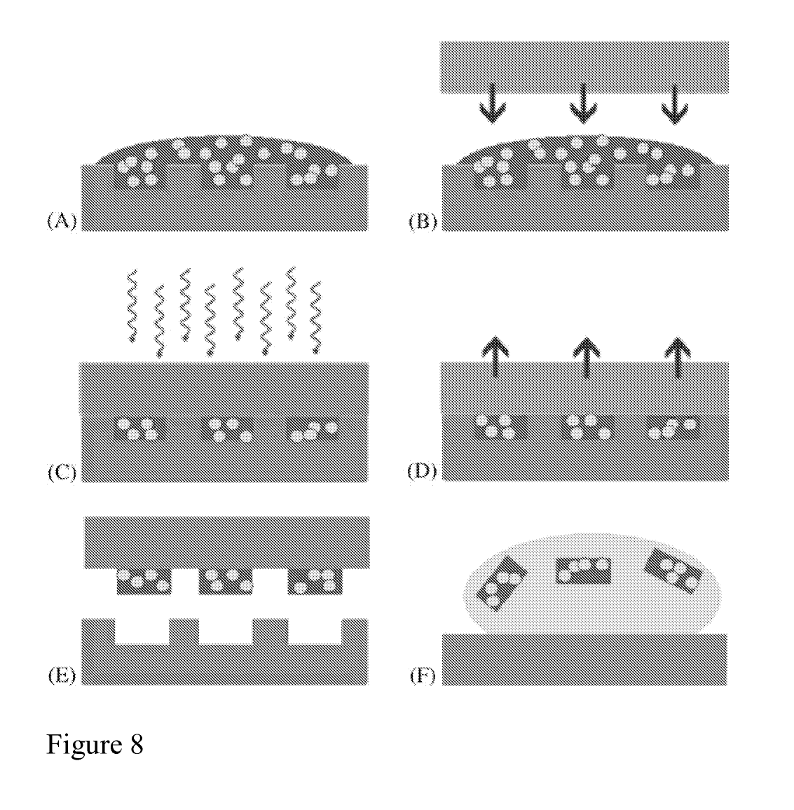

PDMS micropatterns of various shapes were fabricated by curing prepolymer (Sylgard 184, Essex Chemical) on silicon masters patterned with SU-8 photoresist. The patterns on the masters had protruding shapes (e.g. squares, circles, long rectangles, etc.) of various sizes (ranging from 50 μm to 400 μm), which allowed for the formation of shaped wells in PDMS replicas. PDMS molds were generated by pouring 1:10 curing agent to silicon elastomer onto the master and curing for 2 hours at 37° C. Finally, the PDMS molds were peeled from the silicon masters, cut into small rectangular shapes, and placed over glass slides to facilitate ease of manipulation. The use of glass slides allowed direct manipulation of the slides, thereby minimizing possibility of damaging the molds. Before stamping, the molds were rendered hydrophilic by plasma cleaning for 45 seconds on medium power (PDC-001, Harrick Scientific). Untreated (and, therefore, hydrophobic) non-patterned sections of PDMS were similarly placed over glass slides and used as coverslides to reversibly seal the micropatterns into individual volumes (see FIG. 8) during the stamping procedure.

Microgel Polymerization

To suspend NIH/3T3 or murine ES cells within the prepolymer solution, cells were trypsinized with 0.23% trypsin/0.13% EDTA in PBS (Gibco). The suspension was then centrifuged at 1000 rpm for 2 minutes to produce a cell pellet. A volume of the pellet was then resuspended in the prepolymer solution. This yielded differing concentrations of cells in prepolymer solution. 20-25 μl cm⁻² of this cell/polymer mixture was then pipetted onto freshly plasma-oxidized PDMS micropatterns. The tip of the pipette was gently brushed on the micropattern surface to remove any bubbles. A PDMS coverslide was then carefully applied on top of the pattern and gently rotated under slight finger pressure to ensure PDMS-PDMS contact. The micropattern/polymer solution/coverslide assembly was then exposed to approximately 1 W cm⁻² 360 nm-480 nm UV light for various durations. The coverslide was then carefully removed and PBS was immediately pipetted onto the coverslide surface upon which the microgels were adhered to hydrate the newly formed hydrogels.

Microgel Harvesting

After photopolymerization, the coverslide was removed from the microwell substrate to retrieve the microgels. In this process, a fraction of the microgels adhered to the microwell surface while the other fraction adhered to the PDMS coverslide. For convenience, those microgels which adhered to the PDMS coverslide upon removal of the coverslide from the PDMS micropattern were then harvested while those which remained adhered within the microwells were discarded. After hydrating the microgels upon the coverslide, a number of individual microgels spontaneously detached from the coverslide while a number remained adhered. A pipette tip was gently brushed over the coverslide to mechanically detach the remaining microgels.

Analysis of Encapsulated Cells

Initial encapsulated cell viability was assessed by applying a live/dead fluorescence assay to a model polymer system. In this system, cells encapsulated in thin layers of HA hydrogels were made by deposition of 20 μl cell/prepolymer mixture between a glass slide and a flat PDMS coverslide. After photopolymerization, the PDMS coverslide was removed, leaving a thin layer of polymer/cell adhered to the glass slide. The thin layer was subsequently hydrated with 200 μl PBS solution containing 2 μg/ml calcein AM and 4 μg/ml ethidium homodimer-1 (Molecular Probes) and visualized under a fluorescent microscope (Zeiss, Axiovert 200). Initial cell viability assessments were made using NIH-3T3 cells for MeHA prepolymer solutions in which the macromer concentration, UV exposure duration, and photoinitiator concentrations were varied.

Two recognized parameters of cell viability—intracellular esterase activity and plasma membrane integrity—were tracked. Live cells fluoresced green, showing intracellular esterase activity that hydrolyzed the fluorogenic esterase substrate (calcein AM) to a green fluorescent product, and dead cells fluoresced red, their plasma membrane being compromised and therefore permeable to the high-affinity, red fluorescent nucleic acid stain (ethidium homodimer-1). Percent viability values were calculated by counting the number of live (green) cells and the number of dead (red) cells in a representative 400 μm×400 μm square area magnified at 50× and dividing the number of live cells by the number of total cells (live plus dead). Measurements were taken in triplicates, and error bars were based on standard deviation values for n=3.

For confocal microscopy, cells were stained with Vybrant DiD (Molecular Probes) at 20 μl/ml in PBS, fixed with Fluoromount-G, and covered with a No. 1 thickness coverslip. Confocal images were taken at 40× magnifications through a Rhodamine filter with a maximum focal depth of 248 μm. CFSE and PKH26 staining were performed as previously described (Khademhosseini et al., 2004, Biomaterials, 25:3583) at room temperature prior to microscopy.

### Results

Microgel Fabrication

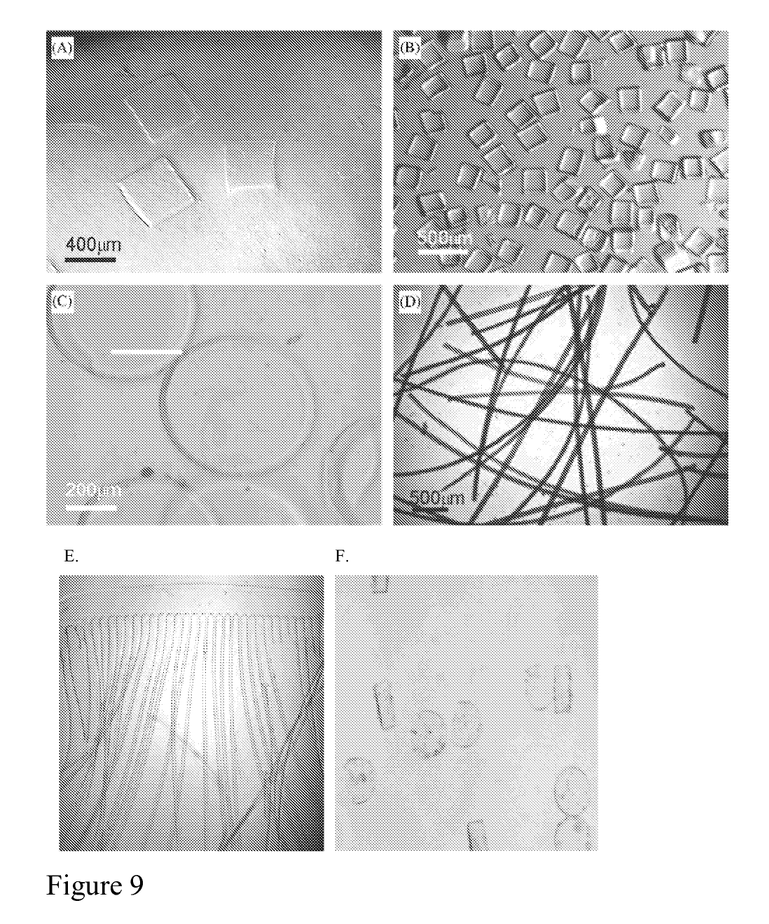

The procedure illustrated in FIG. 8 was used to fabricate homogenous microgels of specific shapes and sizes. In this process cells were suspended in a hydrogel precursor solution and molded using a PDMS stamp. Subsequently, the hydrogel precursor solution was induced to photocrosslink to form a gel. The mold was then removed to generate an array of micromolded hydrogels that could be harvested into the solution using a simple wash. The techniques can be used to generate microgel suspensions of virtually any shape as long as the desired pattern can be fabricated in the PDMS molds. Proof-of-concept square prisms, disks, and strings were fabricated with high pattern fidelity (FIG. 9). In addition, both MeHA and PEGDA polymer solutions were successfully used to fabricate hydrogels suggesting that the micromolding techniques is potentially compatible with other hydrogels such as collagen, agarose, and dextran.

PDMS molds containing negative patterns of desired shapes were made using soft lithographic techniques, and prepolymer mixtures were photopolymerized within the corresponding positive patterns. In order to assess the effect of microgel concentration on the formation of a hydrogel and its resulting mechanical properties, a series of macromer concentrations (i.e. hydrogel precursor solution) were tested. It was observed that the properties of the hydrogels could be, significantly altered based on the polymer concentration. For example, different concentrations of MeHA macromer in PBS (2%, 5%, 7.5%, 10% w/w) were tested, and it was found that the uptake of PBS solution and the subsequent swelling of the MeHA microgels increased with higher macromer concentrations. Also, the mechanical robustness of the microgels increased with increasing macromer concentrations. In particular, mechanical stimulation of MeHA microgels of low macromer concentration tended to break them into debris while the microgels of higher macromer concentration remained relatively intact. In addition, it was noted that the larger microgels swelled more upon hydration. Without wishing to be bound by any one theory, this phenomenon may occur because the swelling ratio of the gels was proportional to the volume of the gels, which scales as a cubic function of length, while the surface area of the gels scales as a square function of length.

Optimization for Cell Viability

In order to optimize the various parameters (e.g. macromer concentration, photoinitiator concentration, UV length, etc.) for cell viability prior to encapsulating cells within microgels, initial cell viability data were obtained through the utilization of a thin-layer model. The setup comprised cells encapsulated in thin layers of HA hydrogels sandwiched between a glass slide and a flat PDMS coverslide. Following photopolymerization and subsequent removal of the PDMS coverslide, a thin layer of polymer/cell solution was formed on the glass slide, allowing easy access for the application of viability assay and visualization under fluorescent microscopy.

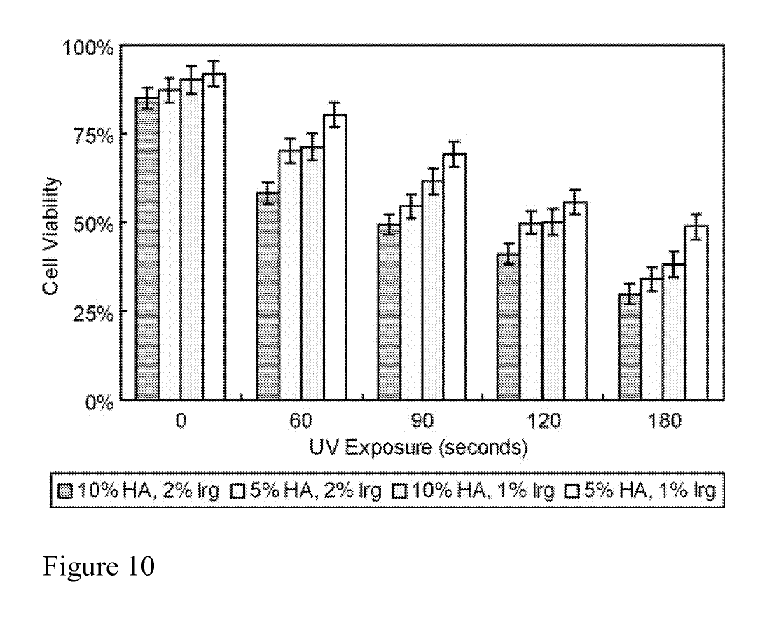

It is known that photoinitiator concentration, UV exposure length, and macromer concentration may affect the viability of cells encapsulated within photopolymerized hydrogels (Burdick et al., 2005, Biomacromolecules, 6:386). Therefore, these parameters were varied and the cell viability was analyzed. As expected, cell viability decreased with increasing UV exposure length for all photoinitiator and macromer concentration conditions (FIG. 10). Furthermore, increased photoinitator and macromer concentrations individually and in combination also decreased cell viability. Based on these results, 5% MeHA in PBS with 1% photoinitiator UV-exposed for 60 seconds was determined to be optimal for maintaining high cell viability within hydrogel films for this particular set of experiments. One of ordinary skill in the art reading this specification will understand how to optimize these conditions for different experimental setups. The identity of these optimal conditions is consistent with what is known in the art; cell viability is typically higher for lower macromer concentrations, lower photoinitiator concentration, and shorter UV exposure lengths).

Cell Encapsulation Within Microgels

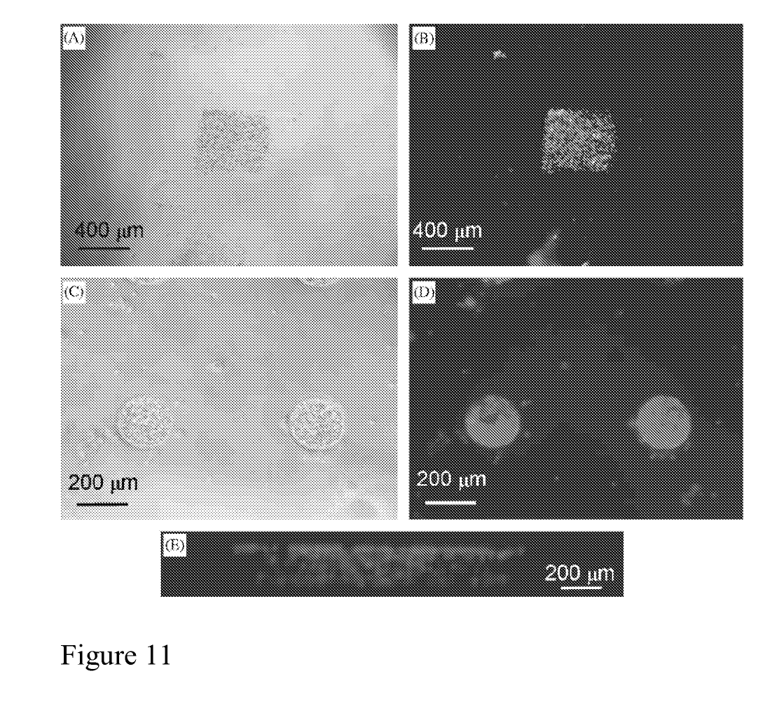

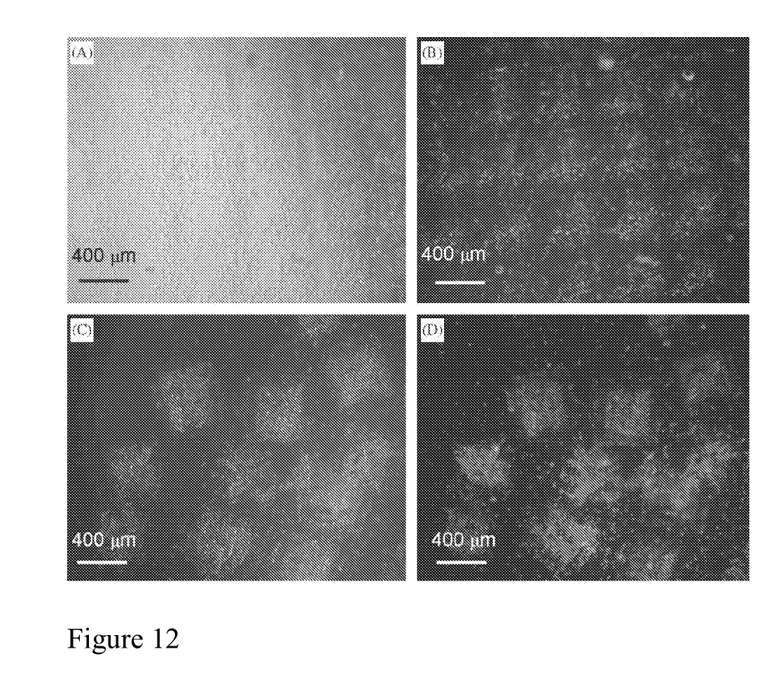

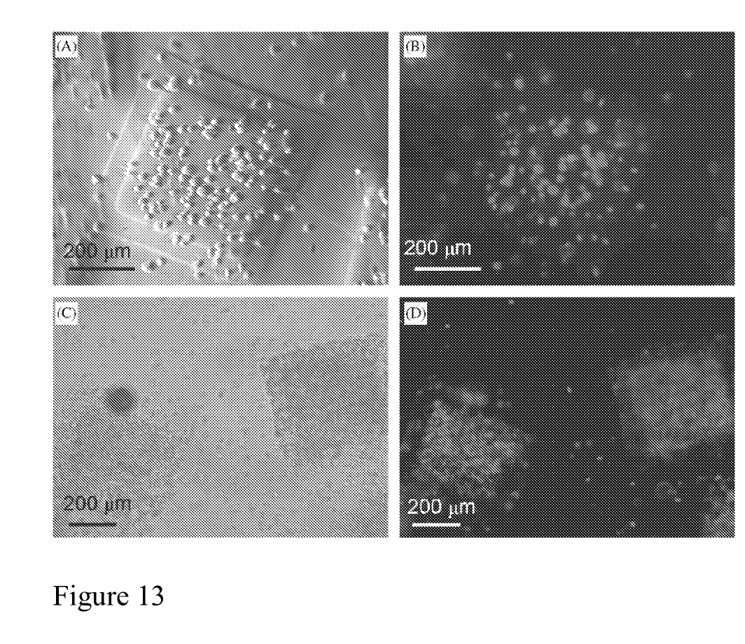

Using optimized parameters found through the thin-layer model as a starting point, cells were successfully encapsulated within microgels and shown to be viable (>85%) after the photocrosslinking step (FIG. 11). Uniform cell distribution was demonstrated throughout the depth of individual microgels (FIG. 11E) as well as across different microgels (FIG. 12A), and easy retrieval of these microgels was achieved through the subsequent hydration and suspension step (FIG. 12). Moreover, cell density is a parameter that can be finely controlled (FIG. 13), in addition to the size, shape, and uniformity of these microgels.

Additional optimization was performed in order to accommodate the encapsulation of cells within microgels. It was found, for example, that the introduction of cells led to weaker mechanical stability of the gels for any given macromer concentration, photoinitiator concentration, and UV exposure duration, and that the degree of compromise was proportional to the density of cells encapsulated. In order to counter the higher tendency for debris formation, the MeHA concentration was increased from 5 wt %-10 wt %, which in turn allowed the UV exposure duration to be reduced from 60 seconds to 45 seconds.

Another challenge was that a thin film tended to form between the PDMS coverslide and the PDMS pattern upon photopolymerization of prepolymer/cell mixture, especially at high cell densities. The thin film inhibited harvesting of the microgels because the microgels were then covalently attached to the thin film and could not be removed. Without wishing to be bound by any one theory, this may be due to the cells which did not fall into the molds being sandwiched between the coverslide and non-well areas of the pattern pieces, thereby preventing complete sealing between the coverslide and pattern. The phenomenon was minimized by using PDMS patterns with small spacing between the negative features such that the cells could more easily be displaced to the microwells and decreased the likelihood that a cell would be sandwiched between the coverslide and the non-well areas of the pattern pieces. In addition, by maintaining the PDMS coverslip in a hydrophobic state it was possible to maximize dewetting of the solution from the surface and thus minimize the formation of the thin films between microgels. The problem may potentially be reduced by generating microwell patterns with hydrophobic surfaces and hydrophilic wells.

An additional complication of generating photopolymerized microgels in which cells are encapsulated versus microgels without cells is that the solution in which cells are suspended is relatively viscous. When the relatively viscous cell suspension is added to the precursor solution, the viscosity of the precursor solution is increased, which may lead to the difficulties in making microgels that are smaller than 400 μm. Indeed, using PDMS micropatterns of smaller sizes, it was found that hydrogels of the proper shape and size rarely formed when the cell suspension was included in the precursor solution. Post-photopolymerization, it appeared that the precursor solution containing cells did not properly fill the wells and that the wells were, instead, filled with air bubbles. In contrast, using an identical precursor solution except without the cell suspension, microgels formed adequately using PDMS micropatterns as small as 50 μm. In the end, the parameter values of 10% MeHA macromer concentration, 1% photoinitiator, and 45 second UV exposure duration were found to provide good cell viability and microgel stability. These parameter values proved to be amenable for generating microgels with differing encapsulated cell densities (FIG. 13), keeping cells viable past 6 days of incubation in media.

Lastly, cells at the periphery of the microgels were observed to be more likely to lose their viability. This phenomenon can be seen from FIGS. 11-13 in which dead cells (red) were observed around the periphery of the microgels. While not wishing to be bound by any theory, it is possible that these cells were adhered to the surface rather than embedded within the surface layers of the microgel, since they were found to be mobile and detachable from the surface upon physical contact with a micromanipulator.

Hydrogel Arrangements

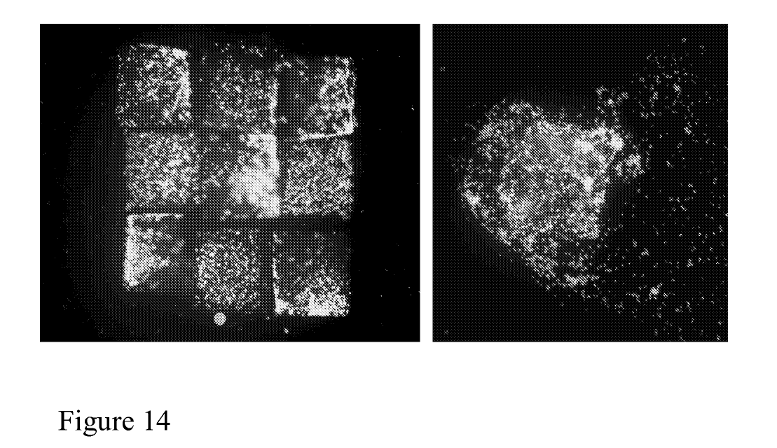

Taking a step beyond easy retrieval, microgels generated using this method can be arranged in specific configurations. An example is an alternating checkerboard pattern (FIG. 14), assembled with fluorescently red- and green-stained cells in separate sets of microgels. This was performed by physically manipulating individual microgels into the pattern using a micromanipulator. Although this approach is time-consuming due to difficulties encountered in manipulating individual microgels, nonetheless, the successful ordered arrangement of shape-specific hydrogels containing different cell types presents the possibility of reproducing physiological cellular arrangements in vitro.

Micromolding could therefore potentially provide a first step to a bottom-up approach to tissue engineering in which individual units of cell-encapsulating microgels are assembled into larger macrostructures of particular three-dimensional configurations. Control over the specific shape and size of microgels may be especially useful since shape-fitting microgels could then be placed adjacent to one another and assembled into larger structures or tissues. In addition, encapsulation of cells within cell-laden hydrogels using micromolding techniques offers several advantages over current systems and can be interfaced with microfluidics technology to engineer the microvasculatrure (Cabodi et al., 2005, J. Am. Chem. Soc., 127:13788).

In summary, a micromolding technique for encapsulating live cells in microscale photocrosslinkable hydrogels of controlled 3D shapes was developed. The distribution of cells successfully encapsulated in both MeHA and PEGDA hydrogels was found to be homogeneous, and the technique was shown to be amenable for various cell densities. This method provides a useful tool for tissue engineering applications that require controlled spatial distribution or architecture of cells.

### Equivalents and Scope

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Thus, for example, reference to “a nanoparticle” includes a plurality of such nanoparticle, and reference to “the cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any hydrogel, any hydrogel assembly, any polymer, any cell type, any crosslinking initiator, any crosslinking method, any method of preparing hydrogels, any method of treatment involving hydrogels, etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

We claim:

1. A hydrogel comprising a crosslinked polymeric network, wherein cells are encapsulated within the hydrogel, and wherein the hydrogel has a greatest dimension ranging between about 1 μm and about 1000 μm.

2. The hydrogel of claim 1, wherein the hydrogel has a greatest dimension ranging between about 50 μm and about 1000 μm.

3. The hydrogel of claim 1, wherein the hydrogel has a greatest dimension ranging between about 100 μm and about 1000 μm.

4. The hydrogel of claim 1, wherein the hydrogel has a greatest dimension of about 50 μm.

5. The hydrogel of claim 1, wherein the hydrogel has a greatest dimension of about 100 μm.

6. The hydrogel of claim 1, wherein the hydrogel has a greatest dimension of about 250 μm.

7. The hydrogel of claim 1, wherein the hydrogel has a greatest dimension of about 500 μm.

8. The hydrogel of claim 1, wherein the hydrogel has a greatest dimension of about 750 μm.

9. The hydrogel of claim 1, wherein the hydrogel has a greatest dimension of about 1000 μm.

10. The hydrogel of claim 1, wherein the cells are distributed evenly throughout the hydrogel.

11. The hydrogel of claim 1, wherein the cells are selected from the group consisting of cardiomyocytes, myocytes, hepatocytes, keratinocytes, melanocytes, neurons, astrocytes, embryonic stem cells, adult stem cells, hematopoietic stem cells, hematopoietic cells, monocytes, neutrophils, macrophages, ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, and hybrids thereof.

12. The hydrogel of claim 1, wherein the hydrogel comprises an environment that promotes cell viability.

13. The hydrogel of claim 1, wherein the hydrogel comprises one type of cell.

14. The hydrogel of claim 1, wherein the hydrogel comprises more than one type of cell.

15. The hydrogel of claim 1, wherein the polymeric network comprises a natural polymer.

16. The hydrogel of claim 1, wherein the polymeric network comprises a carbohydrate.

17. The hydrogel of claim 1, wherein the polymeric network comprises a carbohydrate selected from the group consisting of hyaluronic acid (HA), chondroitin sulphate, dermatan sulphate, keratan sulphate, heparan sulphate, derivatives thereof, and combinations thereof.

18. The hydrogel of claim 1, wherein the polymeric network comprises a carbohydrate selected from the group consisting of alginate, chitosan, agarose, heparin, dextran, derivatives thereof, and combinations thereof.

19. The hydrogel of claim 1, wherein the polymeric network comprises a protein or peptide.

20. The hydrogel of claim 1, wherein the polymeric network comprises a protein selected from the group consisting of collagen, elastin, fibrin, derivatives thereof, and combinations thereof.

21. The hydrogel of claim 1, wherein the polymeric network comprises a synthetic polymer.

22. The hydrogel of claim 1, wherein the polymeric network comprises a polymer selected from the group consisting of poly(ethylene glycol) (PEG), poly(hydroxyethyl methacrylate) (PHEMA), poly(vinyl alcohol) (PVA), derivatives thereof, and combinations thereof.

23. The hydrogel of claim 1, wherein the polymeric network comprises one type of polymer.

24. The hydrogel of claim 1, wherein the polymeric network comprises more than one type of polymer.

25. The hydrogel of claim 1, wherein the polymeric network is crosslinked using a chemical crosslinking agent.

26. The hydrogel of claim 25, wherein the polymeric network is crosslinked using a chemical crosslinking agent selected from the group consisting of electrophiles and nucleophiles.

27. The hydrogel of claim 25, wherein the polymeric network is crosslinked using a chemical crosslinking agent selected from the group consisting of glutaraldehyde, acetaldehyde, formaldehyde, derivatives thereof, and combinations thereof.

28. The hydrogel of claim 1, wherein the polymeric network is crosslinked by altering pH such that crosslinking occurs.

29. The hydrogel of claim 1, wherein the polymeric network is crosslinked by an ionic crosslinking mechanism.

30. The hydrogel of claim 29, wherein the polymeric network is crosslinked due to formation of ionic bridges between divalent cations and the polymer.

31. The hydrogel of claim 29, wherein the polymeric network is crosslinked due to interactions between cations and negatively charged functional groups.

32. The hydrogel of claim 1, wherein the polymeric network is crosslinked by a physical crosslinking mechanism.

33. The hydrogel of claim 32, wherein the polymeric network is crosslinked by performing repeated cycles of freezing and thawing.

34. The hydrogel of claim 1, wherein the polymeric network is crosslinked using irradiative crosslinking mechanisms.

35. The hydrogel of claim 34, wherein the polymeric network is crosslinked using electron beam irradiation, gamma irradiation, or combinations thereof.

36. The hydrogel of claim 1, wherein the polymeric network is crosslinked using photocrosslinking mechanisms.

37. The hydrogel of claim 36, wherein the polymeric network is crosslinked by a photoinitiator and ultraviolet (UV) light.

38. The hydrogel of claim 37, wherein the photoinitiator is 2-hydroxy-1-(4-(hydroxyethoxy)phenyl)-2-methyl-1-propanone.

39. The hydrogel of claim 1, wherein the polymeric network is crosslinked using thermal crosslinking mechanisms.

40. The hydrogel of claim 1, wherein the polymeric network is crosslinked by a thermal initiator and heat.

41. The hydrogel of claim 40, wherein the thermal initiator is selected from the group consisting of peroxides, peracids, peracetates, and persulfates.

42. The hydrogel of claim 1, wherein a therapeutic agent to be delivered is encapsulated within the hydrogel.

43. The hydrogel of claim 1, wherein the hydrogel is biocompatible, biodegradable, or both.

44. The hydrogel of claim 1, wherein the hydrogel is characterized by a cuboid, rectangular, spherical, conical, pyramid-like, cylindrical, tubular, ring-shaped, tetrahedral, hexagonal, or octagonal shape.

45. A hydrogel assembly comprising a plurality of hydrogels, wherein hydrogel comprise a crosslinked polymeric network, wherein cells are encapsulated within hydrogels, and wherein hydrogels has a greatest dimension ranging between about 1 μm and about 1000 μm.

46. A method comprising:
providing a suspension of cells;
providing a polymer capable of forming hydrogels;
mixing the suspension of cells and the polymer to form a precursor solution;
pouring the precursor solution into stamps, wherein the stamps comprise a plurality of wells and ridges;
crosslinking the precursor solution inside the molds such that hydrogels are formed, wherein the hydrogels have a greatest dimension ranging between about 1 μm and about 1000 μm; and
harvesting the hydrogels.

47. The method of claim 46, wherein the concentration of cells within the precursor solution ranges between about 0.1% and about 80%.

48. The method of claim 46, wherein the concentration of cells within the precursor solution ranges between about 1.0% and about 50%.

49. The method of claim 46, wherein the concentration of cells within the precursor solution ranges between about 1.0% and about 20%.

50. The method of claim 46, wherein the concentration of cells within the precursor solution ranges between about 1.0% and about 10%.

51. The method of claim 46, wherein the concentration of cells within the precursor solution is about 5%.

52. The method of claim 46, wherein the concentration of polymer within the precursor solution ranges between about 1% and about 40%.

53. The method of claim 46, wherein the concentration of polymer within the precursor solution ranges between about 1% and about 20%.

54. The method of claim 46, wherein the concentration of polymer within the precursor solution ranges between about 1% and about 10%.

55. The method of claim 46, wherein the concentration of polymer within the precursor solution is about 5%.

56. The method of claim 46, wherein the precursor solution further comprises a chemical crosslinking agent, photoinitiator, thermal initiator, or combinations thereof.

57. The method of claim 46, wherein the stamps comprise poly(dimethylsiloxane) (PDMS).

58. The method of claim 57, wherein the surfaces of the PDMS stamps are made to be hydrophilic by plasma cleaning.

59. The method of claim 46, wherein the hydrogels are harvested by rinsing the stamps with an aqueous solution, water-miscible solvent, or combination thereof.

60. The method of claim 46, further comprising steps of:
assembling the hydrogels such that a hydrogel assembly is formed; and
performing a second crosslinking step, wherein the second crosslinking step is performed in order to crosslink individual hydrogels to one another.

61. A method, comprising:
providing a plurality of microgels;
assembling the plurality of microgels such that a hydrogel assembly is formed.

62. The method of claim 61, wherein all of the microgels of the plurality of microgels are identical to one another.

63. The method of claim 61, wherein all of the microgels of the plurality of microgels are not identical to one another.

64. The method of claim 61, wherein the hydrogel assembly is formed by a self-assembly mechanism.

65. The method of claim 61, wherein microgel surfaces are modified in a way that promotes self-assembly.

66. The method of claim 65, wherein modified microgel surfaces can interact with one another via hydrophobic forces, electrostatic forces, polymer chain entanglement, or affinity interactions.

67. The method of claim 65, wherein microgel surfaces are modified with one or more functional groups.

68. The method of claim 67, wherein the functional group is selected from the group consisting of carboxyl groups, amino groups, and hydroxyl groups.

69. The method of claim 68, wherein microgel surfaces are modified with two or more substances that specifically bind to one another with high affinity.

70. The method of claim 69, wherein microgel surfaces are modified with proteins, nucleic acids, lipids, carbohydrates, metals, small molecules, or drugs which are capable of participating in affinity interactions, such that individual microgels bind to one another via affinity interactions.

71. The method of claim 65, wherein microgel shape is controlled in a way that promotes self-assembly via a geometric lock-and-key mechanism.

72. The method of claim 65, wherein hydrogel assemblies are formed utilizing surface tension mechanisms.

73. The method of claim 72, wherein surface tension mechanisms involve placing individual microgels within a hydrophobic bath such that a two-phase system is developed, wherein each microgel is separated from the oil phase by a surface tension, and wherein surface tension is minimized when the microgels self-assemble.

74. The method of claim 61, wherein the hydrogel assembly is formed manually.

75. The method of claim 74, wherein the hydrogel assembly is formed manually using a micromanipulator.

76. The method of claim 61, further comprising an additional crosslinking step performed after hydrogel assembly has occurred, wherein the additional crosslinking step is performed in order to crosslink individual microgels to one another.

77. A method comprising:
providing a subject in need of tissue regeneration; and
administering one or more cell-laden hydrogels or cell-laden hydrogel assemblies to the subject in order to promote tissue regeneration.

78. A method comprising:
providing a subject having a location in need of tissue regeneration; and
providing a precursor solution comprising cells and at least one polymer capable of forming a hydrogel;
administering the precursor solution in situ to the subject at or near the location in need of tissue regeneration; and
crosslinking the precursor solution in situ such that a hydrogel is formed at or near the location in need of tissue regeneration.
